SEQUENCE LISTING (25 sequences)

SEQ ID NO: 1 - protein, 480 aa, Staphylococcus aureus
AYTVTKPQTTQTVSKIAQVKPNNTGIRASVYEKTAKNGAKYADRTFYVTKERAHGNETYVLLNNTSHNIPLGWFNVKDLNVQNLGKEVKTTQKYTVNKSNNGLSMVPWGTKNQVILTGNNIAQGTFNATKQVSVGKDVYLYGTINNRTGWVNAKDLTAPTAVKPTTSAAKDYNYTYVIKNGNGYYYVTPNSDTAKYSLKAFNEQPFAVVKEQVINGQTWYYGKLSNGKLAWIKSTDLAKELIKYNQTGMTLNQVAQIQAGLQYKPQVQRVPGKWTDANFNDVKHAMDTKRLAQDPALKYQFLRLDQPQNISIDKINQFLKGKGVLENQGAAFNKAAQMYGINEVYLISHALLETGNGTSQLAKGADVVNNKVVTNSNTKYHNVFGIAAYDNDPLREGIKYAKQAGWDTVSKAIVGGAKFIGNSYVKAGQNTLYKMRWNPAHPGTHQYATDVDWANINAKIIKGYYDKIGEVGKYFDIPQY

SEQ ID NO: 2 - DNA, 3771 bp, Staphylococcus aureus
atggcgaaaaaattcaattacaaactaccatcaatggttgcattaacgcttgtaggttcagcagtcactgcacatcaagttcaagcagctgagacgacacaagatcaaactactaataaaaacgttttagatagtaataaagttaaagcaactactgaacaagcaaaagctgaggtaaaaaatccaacgcaaaacatttctggcactcaagtatatcaagaccctgctattgtccaaccaaaaacagcaaataacaaaacaggcaatgctcaagtaagtcaaaaagttgatactgcacaagtaaatggtgacactcgtgctaatcaatcagcgactacaaataatacgcagcctgttgcaaagtcaacaagcactacagcacctaaaactaacactaatgttacaaatgctggttatagtttagttgatgatgaagatgataattcagaaaatcaaattaatccagaattaattaaatcagctgctaaacctgcagctcttgaaacgcaatataaaaccgcagcacctaaagctgcaactacatcagcacctaaagctaaaactgaagcgacacctaaagtaactacttttagcgcttcagcacaaccaagatcagttgctgcaacaccaaaaacgagtttgccaaaatataaaccacaagtaaactcttcaattaacgattacattcgtaaaaataacttaaaagcacctaaaattgaagaagattatacatcttacttccctaaatacgcataccgtaacggcgtaggtcgtcctgaaggtatcgtagttcatgatacagctaatgatcgttcgacgataaatggtgaaattagttatatgaaaaataactatcaaaacgcattcgtacatgcatttgttgatggggatcgtataatcgaaacagcaccaacggattacttatcttggggtgtcggtgcagtcggtaaccctagattcatcaatgttgaaatcgtacacacacacgactatgcttcatttgcacgttcaatgaataactatgctgactatgcagctacacaattacaatattatggtttaaaaccagacagtgctgagtatgatggaaatggtacagtatggactcactacgctgtaagtaaatatttaggtggtactgaccatgccgatccacatggatatttaagaagtcataattatagttatgatcaattatatgacttaattaatgaaaaatatttaataaaaatgggtaaagtggcgccatggggtacgcaatctacaactacccctactacaccatcaaaaccaacaacaccgtcgaaaccatcaactggtaaattaacagttgctgcaaacaatggtgtcgcacaaatcaaaccaacaaatagtggtttatatactactgtatacgacaaaactggtaaagcaactaatgaagttcaaaaaacatttgctgtatctaaaacagctacattaggtaatcaaaaattctatcttgttcaagattacaattctggtaataaatttggttgggttaaagaaggcgatgtggtttacaacacagctaaatcacctgtaaatgtaaatcaatcatattcaatcaaacctggtacgaaactttatacagtaccttggggtacatctaaacaagttgctggtagtgtgtctggctctggaaaccaaacatttaaggcttcaaagcaacaacaaattgataaatcaatttatttatatggctctgtgaatggtaaatctggttgggtaagtaaagcatatttagttgatactgctaaacctacgcctacaccaacacctaagccatcaacacctacaacaaataataaattaacagtttcatcattaaacggtgttgctcaaattaatgctaaaaacaatggcttattcactacagtttatgacaaaactggtaagccaacgaaagaagttcaaaaaacatttgctgtaacaaaagaagcaagtttaggtggaaacaaattctacttagttaaagattacaatagtccaactttaattggttgggttaaacaaggtgacgttatttataacaatgcaaaatcacctgtaaatgtaatgcaaacatatacagtaaaaccaggcactaaattatattcagtaccttggggcacttataaacaagaagctggtgcagtttctggtacaggtaaccaaacttttaaagcgactaagcaacaacaaattgataaatctatctatttatttggaactgtaaatggtaaatctggttgggtaagtaaagcatatttagctgtacctgctgcacctaaaaaagcagtagcacaaccaaaaacagctgtaaaagcttatactgttactaaaccacaaacgactcaaacagttagcaagattgctcaagttaaaccaaacaacactggtattcgtgcttctgtttatgaaaaaacagcgaaaaacggtgcgaaatatgcagaccgtacgttctatgtaacaaaagagcgtgctcatggtaatgaaacgtatgtattattaaacaatacaagccataacatcccattaggttggttcaatgtaaaagacttaaatgttcaaaacttaggcaaagaagttaaaacgactcaaaaatatactgttaataaatcaaataacggcttatcaatggttccttggggtactaaaaaccaagtcattttaacaggcaataacattgctcaaggtacatttaatgcaacgaaacaagtatctgtaggcaaagatgtttatttatacggtactattaataaccgcactggttgggtaaatgcaaaagatttaactgcaccaaccgctgtgaaaccaactacatcagctgccaaagattataactacacttatgtaattaaaaatggtaatggttattactatgtaacaccaaattctgatacagctaaatactcattaaaagcatttaatgaacaaccattcgcagttgttaaagaacaagtcattaatggacaaacttggtactatggtaaattatctaacggtaaattagcatggattaaatcaactgatttagctaaagaattaattaagtataatcaaacaggtatgacattaaaccaagttgctcaaatacaagctggtttacaatataaaccacaagtacaacgtgtaccaggtaagtggacagatgctaactttaatgatgttaagcatgcaatggatacgaagcgtttagctcaagatccagcattaaaatatcaattcttacgcttagaccaaccacaaaatatttctattgataaaattaatcaattcttaaaaggtaaaggtgtattagaaaaccaaggtgctgcatttaacaaagctgctcaaatgtatggcattaatgaagtttatcttatctcacatgccctattagaaacaggtaacggtacttctcaattagcgaaaggtgcagatgtagtgaacaacaaagttgtaactaactcaaacacgaaataccataacgtatttggtattgctgcatatgataacgatcctttacgtgaaggtattaaatatgctaaacaagctggttgggacacagtatcaaaagcaatcgttggtggtgctaaattcatcggcaactcatatgtaaaagctggtcaaaatacactttacaaaatgagatggaatcctgcacatccaggaacacaccaatatgctacagatgtagattgggctaacatcaatgctaaaatcatcaaaggctactatgataaaattggcgaagtcggcaaatacttcgacatcccacaatataaataa

SEQ ID NO: 3 - protein, 578 aa, Staphylococcus aureus
SASAQPRSVAATPKTSLPKYKPQVNSSINDYIRKNNLKAPKIEEDYTSYFPKYAYRNGVGRPEGIVVHDTANDRSTINGEISYMKNNYQNAFVHAFVDGDRIIETAPTDYLSWGVGAVGNPRFINVEIVHTHDYASFARSMNNYADYAATQLQYYGLKPDSAEYDGNGTVWTHYAVSKYLGGTDHADPHGYLRSHNYSYDQLYDLINEKYLIKMGKVAPWGTQSTTTPTTPSKPTTPSKPSTGKLTVAANNGVAQIKPTNSGLYTTVYDKTGKATNEVQKTFAVSKTATLGNQKFYLVQDYNSGNKFGWVKEGDVVYNTAKSPVNVNQSYSIKPGTKLYTVPWGTSKQVAGSVSGSGNQTFKASKQQQIDKSIYLYGSVNGKSGWVSKAYLVDTAKPTPTPTPKPSTPTTNNKLTVSSLNGVAQINAKNNGLFTTVYDKTGKPTKEVQKTFAVTKEASLGGNKFYLVKDYNSPTLIGWVKQGDVIYNNAKSPVNVMQTYTVKPGTKLYSVPWGTYKQEAGAVSGTGNQTFKATKQQQIDKSIYLFGTVNGKSGWVSKAYLAVPAAPKKAVAQPKTAVK

SEQ ID NO: 4 - protein, 350 aa, Staphylococcus aureus
MTKHYLNSKYQSEQRSSAMKKITMGTASIILGSLVYIGADSQQVNAATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 5 - DNA, 1053 bp, Staphylococcus aureus
atgacaaaacattatttaaacagtaagtatcaatcagaacaacgttcatcagctatgaaaaagattacaatgggtacagcatctatcattttaggttcccttgtatacataggcgcagacagccaacaagtcaatgcggcaacagaagctacgaacgcaactaataatcaaagcacacaagtttctcaagcaacatcacaaccaattaatttccaagtgcaaaaagatggctcttcagagaagtcacacatggatgactatatgcaacaccctggtaaagtaattaaacaaaataataaatattatttccaaaccgtgttaaacaatgcatcattctggaaagaatacaaattttacaatgcaaacaatcaagaattagcaacaactgttgttaacgataataaaaaagcggatactagaacaatcaatgttgcagttgaacctggatataagagcttaactactaaagtacatattgtcgtgccacaaattaattacaatcatagatatactacgcatttggaatttgaaaaagcaattcctacattagctgacgcagcaaaaccaaacaatgttaaaccggttcaaccaaaaccagctcaacctaaaacacctactgagcaaactaaaccagttcaacctaaagttgaaaaagttaaacctactgtaactacaacaagcaaagttgaagacaatcactctactaaagttgtaagtactgacacaacaaaagatcaaactaaaacacaaactgctcatacagttaaaacagcacaaactgctcaagaacaaaataaagttcaaacacctgttaaagatgttgcaacagcgaaatctgaaagcaacaatcaagctgtaagtgataataaatcacaacaaactaacaaagttacaaaacataacgaaacgcctaaacaagcatctaaagctaaagaattaccaaaaactggtttaacttcagttgataactttattagcacagttgccttcgcaacacttgcccttttaggttcattatctttattacttttcaaaagaaaagaatctaaataa

SEQ ID NO: 6 - protein, 645 aa, Staphylococcus aureus
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 7 - DNA, 1938 bp, Staphylococcus aureus
atgaacaaacagcaaaaagaatttaaatcattttattcaattagaaagtcatcactaggcgttgcatctgtagcaattagtacacttttattattaatgtcaaatggcgaagcacaagcagcagctgaagaaacaggtggtacaaatacagaagcacaaccaaaaactgaagcagttgcaagtccaacaacaacatctgaaaaagctccagaaactaaaccagtagctaatgctgtctcagtatctaataaagaagttgaggcccctacttctgaaacaaaagaagctaaagaagttaaagaagttaaagcccctaaggaaacaaaagaagttaaaccagcagcaaaagccactaacaatacatatcctattttgaatcaggaacttagagaagcgattaaaaaccctgcaataaaagacaaagatcatagcgcaccaaactctcgtccaattgattttgaaatgaaaaagaaagatggaactcaacagttttatcattatgcaagttctgttaaacctgctagagttattttcactgattcaaaaccagaaattgaattaggattacaatcaggtcaattttggagaaaatttgaagtttatgaaggtgacaaaaagttgccaattaaattagtatcatacgatactgttaaagattatgcttacattcgcttctctgtatcaaacggaacaaaagctgttaaaattgttagttcaacacacttcaataacaaagaagaaaaatacgattacacattaatggaattcgcacaaccaatttataacagtgcagataaattcaaaactgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattattagctttaagtagcatcgttgcattcgtattacctagaaaacgtaaaaactaa

SEQ ID NO: 8 - protein, 895 aa, Staphylococcus aureus
MNKHHPKLRSFYSIRKSTLGVASVIVSTLFLITSQHQAQAAENTNTSDKISENQNNNATTTQPPKDTNQTQPATQPANTAKNYPAADESLKDAIKDPALENKEHDIGPREQVNFQLLDKNNETQYYHFFSIKDPADVYYTKKKAEVELDINTASTWKKFEVYENNQKLPVRLVSYSPVPEDHAYIRFPVSDGTQELKIVSSTQIDDGEETNYDYTKLVFAKPIYNDPSLVKSDTNDAVVTNDQSSSVASNQTNTNTSNQNISTINNANNQPQATTNMSQPAQPKSSTNADQASSQPAHETNSNGNTNDKTNESSNQSDVNQQYPPADESLQDAIKNPAIIDKEHTADNWRPIDFQMKNDKGERQFYHYASTVEPATVIFTKTGPIIELGLKTASTWKKFEVYEGDKKLPVELVSYDSDKDYAYIRFPVSNGTREVKIVSSIEYGENIHEDYDYTLMVFAQPITNNPDDYVDEETYNLQKLLAPYHKAKTLERQVYELEKLQEKLPEKYKAEYKKKLDQTRVELADQVKSAVTEFENVTPTNDQLTDLQEAHFVVFESEENSESVMDGFVEHPFYTATLNGQKYVVMKTKDDSYWKDLIVEGKRVTTVSKDPKNNSRTLIFPYIPDKAVYNAIVKVVVANIGYEGQYHVRIINQDINTKDDDTSQNNTSEPLNVQTGQEGKVADTDVAENSSTATNPKDASDKADVIEPESDVVKDADNNIDKDVQHDVDHLSDMSDNNHFDKYDLKEMDTQIAKDTDRNVDKDADNSVGMSSNVDTDKDSNKNKDKVIQLNHIADKNNHTGKAAKLDVVKQNYNNTDKVTDKKTTEHLPSDIHKTVDKTVKTKEKAGTPSKENKLSQSKMLPKTGETTSSQSWWGLYALLGMLALFIPKFRKESK

SEQ ID NO: 9 - DNA, 2688 bp, Staphylococcus aureus
atgaacaaacatcacccaaaattaaggtctttctattctattagaaaatcaactctaggcgttgcatcggtcattgtcagtacactatttttaattacttctcaacatcaagcacaagcagcagaaaatacaaatacttcagataaaatctcggaaaatcaaaataataatgcaactacaactcagccacctaaggatacaaatcaaacacaacctgctacgcaaccagcaaacactgcgaaaaactatcctgcagcggatgaatcacttaaagatgcaattaaagatcctgcattagaaaataaagaacatgatataggtccaagagaacaagtcaatttccagttattagataaaaacaatgaaacgcagtactatcactttttcagcatcaaagatccagcagatgtgtattacactaaaaagaaagcagaagttgaattagacatcaatactgcttcaacatggaagaagtttgaagtctatgaaaacaatcaaaaattgccagtgagacttgtatcatatagtcctgtaccagaagaccatgcctatattcgattcccagtttcagatggcacacaagaattgaaaattgtttcttcgactcaaattgatgatggagaagaaacaaattatgattatactaaattagtatttgctaaacctatttataacgatccttcacttgtaaaatcagatacaaatgatgcagtagtaacgaatgatcaatcaagttcagtcgcaagtaatcaaacaaacacgaatacatctaatcaaaatatatcaacgatcaacaatgctaataatcaaccgcaggcaacgaccaatatgagtcaacctgcacaaccaaaatcgtcaacgaatgcagatcaagcgtcaagccaaccagctcatgaaacaaattctaatggtaatactaacgataaaacgaatgagtcaagtaatcagtcggatgttaatcaacagtatccaccagcagatgaatcactacaagatgcaattaaaaacccggctatcatcgataaagaacatacagctgataattggcgaccaattgattttcaaatgaaaaatgataaaggtgaaagacagttctatcattatgctagtactgttgaaccagcaactgtcatttttacaaaaacaggaccaataattgaattaggtttaaagacagcttcaacatggaagaaatttgaagtttatgaaggtgacaaaaagttaccagtcgaattagtatcatatgattctgataaagattatgcctatattcgtttcccagtatctaatggtacgagagaagttaaaattgtgtcatctattgaatatggtgagaacatccatgaagactatgattatacgctaatggtctttgcacagcctattactaataacccagacgactatgtggatgaagaaacatacaatttacaaaaattattagctccgtatcacaaagctaaaacgttagaaagacaagtttatgaattagaaaaattacaagagaaattgccagaaaaatataaggcggaatataaaaagaaattagatcaaactagagtagagttagctgatcaagttaaatcagcagtgacggaatttgaaaatgttacacctacaaatgatcaattaacagatttacaagaagcgcattttgttgtttttgaaagtgaagaaaatagtgagtcagttatggacggctttgttgaacatccattctatacagcaactttaaatggtcaaaaatatgtagtgatgaaaacaaaggatgacagttactggaaagatttaattgtagaaggtaaacgtgtcactactgtttctaaagatcctaaaaataattctagaacgctgattttcccatatatacctgacaaagcagtttacaatgcgattgttaaagtcgttgtggcaaacattggttatgaaggtcaatatcatgtcagaattataaatcaggatatcaatacaaaagatgatgatacatcacaaaataacacgagtgaaccgctaaatgtacaaacaggacaagaaggtaaggttgctgatacagatgtagctgaaaatagcagcactgcaacaaatcctaaagatgcgtctgataaagcagatgtgatagaaccagagtctgacgtggttaaagatgctgataataatattgataaagatgtgcaacatgatgttgatcatttatccgatatgtcggataataatcacttcgataaatatgatttaaaagaaatggatactcaaattgccaaagatactgatagaaatgtggataaagatgccgataatagcgttggtatgtcatctaatgtcgatactgataaagactctaataaaaataaagacaaagtcatacagctgaatcatattgccgataaaaataatcatactggaaaagcagcaaagcttgacgtagtgaaacaaaattataataatacagacaaagttactgacaaaaaaacaactgaacatctgccgagtgatattcataaaactgtagataaaacagtgaaaacaaaagaaaaagccggcacaccatcgaaagaaaacaaacttagtcaatctaaaatgctaccaaaaactggagaaacaacttcaagccaatcatggtggggcttatatgcgttattaggtatgttagctttattcattcctaaattcagaaaagaatctaaataa

SEQ ID NO: 10 - protein, 927 aa, Staphylococcus aureus
MNMKKKEKHAIRKKSIGVASVLVGTLIGFGLLSSKEADASENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQSTDASNKDVVNQAVNTSAPRMRAFSLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPEDSDSDPGSDSGSDSNSDSGSDSGSDSTSDSGSDSASDSDSASDSDSASDSDSASDSDSASDSDSDNDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSASDSDSDSDSDSDSDSDSDSDSDSDSDSDSDSDSDSDSDSESDSDSDSDSDSDSDSDSDSDSDSASDSDSGSDSDSSSDSDSESDSNSDSESVSNNNVVPPNSPKNGTNASNKNEAKDSKEPLPDTGSEDEANTSLIWGLLASIGSLLLFRRKKENKDKK

SEQ ID NO: 11 - DNA, 2784 bp, Staphylococcus aureus
atgaatatgaagaaaaaagaaaaacacgcaattcggaaaaaatcgattggcgtggcttcagtgcttgtaggtacgttaatcggttttggactactcagcagtaaagaagcagatgcaagtgaaaatagtgttacgcaatctgatagcgcaagtaacgaaagcaaaagtaatgattcaagtagcgttagtgctgcacctaaaacagacgacacaaacgtgagtgatactaaaacatcgtcaaacactaataatggcgaaacgagtgtggcgcaaaatccagcacaacaggaaacgacacaatcatcatcaacaaatgcaactacggaagaaacgccggtaactggtgaagctactactacgacaacgaatcaagctaatacaccggcaacaactcaatcaagcaatacaaatgcggaggaattagtgaatcaaacaagtaatgaaacgacttctaatgatactaatacagtatcatctgtaaattcacctcaaaattctacaaatgcggaaaatgtttcaacaacgcaagatacttcaactgaagcaacaccttcaaacaatgaatcagctccacagagtacagatgcaagtaataaagatgtagttaatcaagcggttaatacaagtgcgcctagaatgagagcatttagtttagcggcagtagctgcagatgcaccggtagctggcacagatattacgaatcagttgacgaatgtgacagttggtattgactctggtacgactgtgtatccgcaccaagcaggttatgtcaaactgaattatggtttttcagtgcctaattctgctgttaaaggtgacacattcaaaataactgtacctaaagaattaaacttaaatggtgtaacttcaactgctaaagtgccaccaattatggctggagatcaagtattggcaaatggtgtaatcgatagtgatggtaatgttatttatacatttacagactatgtaaatactaaagatgatgtaaaagcaactttgaccatgcccgcttatattgaccctgaaaatgttaaaaagacaggtaatgtgacattggctactggcataggtagtacaacagcaaacaaaacagtattagtagattatgaaaaatatggtaagttttataacttatctattaaaggtacaattgaccaaatcgataaaacaaataatacgtatcgtcagacaatttatgtcaatccaagtggagataacgttattgcgccggttttaacaggtaatttaaaaccaaatacggatagtaatgcattaatagatcagcaaaatacaagtattaaagtatataaagtagataatgcagctgatttatctgaaagttactttgtgaatccagaaaactttgaggatgtcactaatagtgtgaatattacattcccaaatccaaatcaatataaagtagagtttaatacgcctgatgatcaaattacaacaccgtatatagtagttgttaatggtcatattgatccgaatagcaaaggtgatttagctttacgttcaactttatatgggtataactcgaatataatttggcgctctatgtcatgggacaacgaagtagcatttaataacggatcaggttctggtgacggtatcgataaaccagttgttcctgaacaacctgatgagcctggtgaaattgaaccaattccagaggattcagattctgacccaggttcagattctggcagcgattctaattcagatagcggttcagattcgggtagtgattctacatcagatagtggttcagattcagcgagtgattcagattcagcaagtgattcagactcagcgagtgattcagattcagcaagcgattccgactcagcgagcgattccgactcagacaatgactcggattcagatagcgattctgactcagacagtgactcagattccgacagtgactcagattcagatagcgattctgactcagacagtgactcggattcagatagcgattcagattcagatagcgattcagattccgacagtgattccgactcagacagcgattctgactccgacagtgattccgactcagacagcgattcagattccgacagtgattccgactcagatagcgattccgactcagatagcgactcagattcagacagcgattcagattcagacagcgattcagattcagatagcgattcagattccgacagtgactcagattccgacagtgactcggattcagatagcgattcagattccgacagtgactcagattccgacagtgactcagactcagacagtgattcggattcagcgagtgattcggattcagatagtgattccgactccgacagtgactcggattcagatagcgactcagactcggatagcgactcggattcagatagcgattcggactcagatagcgattcagaatcagacagcgattcagattcagacagcgactcagacagtgactcagattcagatagtgactcggattcagcgagtgattcagactcaggtagtgactccgattcatcaagtgattccgactcagaaagtgattcaaatagcgattccgagtcagtttctaacaataatgtagttccgcctaattcacctaaaaatggtactaatgcttctaataaaaatgaggctaaagatagtaaagaaccattaccagatacaggttctgaagatgaagcaaatacgtcactaatttggggattattagcatcaataggttcattactacttttcagaagaaaaaaagaaaataaagataagaaataa

SEQ ID NO: 12 - protein, 877 aa, Staphylococcus aureus
MKKRIDYLSNKQNKYSIRRFTVGTTSVIVGATILFGIGNHQAQASEQSNDTTQSSKNNASADSEKNNMIETPQLNTTANDTSDISANTNSANVDSTTKPMSTQTSNTTTTEPASTNETPQPTAIKNQATAAKMQDQTVPQEANSQVDNKTTNDANSIATNSELKNSQTLDLPQSSPQTISNAQGTSKPSVRTRAVRSLAVAEPVVNAADAKGTNVNDKVTASNFKLEKTTFDPNQSGNTFMAANFTVTDKVKSGDYFTAKLPDSLTGNGDVDYSNSNNTMPIADIKSTNGDVVAKATYDILTKTYTFVFTDYVNNKENINGQFSLPLFTDRAKAPKSGTYDANINIADEMFNNKITYNYSSPIAGIDKPNGANISSQIIGVDTASGQNTYKQTVFVNPKQRVLGNTWVYIKGYQDKIEESSGKVSATDTKLRIFEVNDTSKLSDSYYADPNDSNLKEVTDQFKNRIYYEHPNVASIKFGDITKTYVVLVEGHYDNTGKNLKTQVIQENVDPVTNRDYSIFGWNNENVVRYGGGSADGDSAVNPKDPTPGPPVDPEPSPDPEPEPTPDPEPSPDPEPEPSPDPDPDSDSDSDSGSDSDSGSDSDSESDSDSDSDSDSDSDSDSESDSDSESDSESDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSRVTPPNNEQKAPSNPKGEVNHSNKVSKQHKTDALPETGDKSENTNATLFGAMMALLGSLLLFRKRKQDHKEKA

SEQ ID NO: 13 - DNA, 2634 bp, Staphylococcus aureus
ttgaaaaaaagaattgattatttgtcgaataagcagaataagtattcgattagacgttttacagtaggtaccacatcagtaatagtaggggcaactatactatttgggataggcaatcatcaagcacaagcttcagaacaatcgaacgatacaacgcaatcttcgaaaaataatgcaagtgcagattccgaaaaaaacaatatgatagaaacacctcaattaaatacaacggctaatgatacatctgatattagtgcaaacacaaacagtgcgaatgtagatagcacaacaaaaccaatgtctacacaaacgagcaataccactacaacagagccagcttcaacaaatgaaacacctcaaccgacggcaattaaaaatcaagcaactgctgcaaaaatgcaagatcaaactgttcctcaagaagcaaattctcaagtagataataaaacaacgaatgatgctaatagcatagcaacaaacagtgagcttaaaaattctcaaacattagatttaccacaatcatcaccacaaacgatttccaatgcgcaaggaactagtaaaccaagtgttagaacgagagctgtacgtagtttagctgttgctgaaccggtagtaaatgctgctgatgctaaaggtacaaatgtaaatgataaagttacggcaagtaatttcaagttagaaaagactacatttgaccctaatcaaagtggtaacacatttatggcggcaaattttacagtgacagataaagtgaaatcaggggattattttacagcgaagttaccagatagtttaactggtaatggagacgtggattattctaattcaaataatacgatgccaattgcagacattaaaagtacgaatggcgatgttgtagctaaagcaacatatgatatcttgactaagacgtatacatttgtctttacagattatgtaaataataaagaaaatattaacggacaattttcattacctttatttacagaccgagcaaaggcacctaaatcaggaacatatgatgcgaatattaatattgcggatgaaatgtttaataataaaattacttataactatagttcgccaattgcaggaattgataaaccaaatggcgcgaacatttcttctcaaattattggtgtagatacagcttcaggtcaaaacacatacaagcaaacagtatttgttaaccctaagcaacgagttttaggtaatacgtgggtgtatattaaaggctaccaagataaaatcgaagaaagtagcggtaaagtaagtgctacagatacaaaactgagaatttttgaagtgaatgatacatctaaattatcagatagctactatgcagatccaaatgactctaaccttaaagaagtaacagaccaatttaaaaatagaatctattatgagcatccaaatgtagctagtattaaatttggtgatattactaaaacatatgtagtattagtagaagggcattacgacaatacaggtaagaacttaaaaactcaggttattcaagaaaatgttgatcctgtaacaaatagagactacagtattttcggttggaataatgagaatgttgtacgttatggtggtggaagtgctgatggtgattcagcagtaaatccgaaagacccaactccagggccgccggttgacccagaaccaagtccagacccagaaccagaaccaacgccagatccagaaccaagtccagacccagaaccggaaccaagcccagacccggatccggattcggattcagacagtgactcaggctcagacagcgactcaggttcagatagcgactcagaatcagatagcgattcggattcagacagtgattcagattcagacagcgactcagaatcagatagcgactcagaatcagatagtgagtcagattcagacagtgactcggactcagacagtgattcagactcagatagcgattcagactcagatagcgattcagactcagacagcgattcagattcagacagcgactcagattcagacagcgactcagactcagatagcgactcagactcagacagcgactcagattcagatagcgattcagactcagacagcgactcagactcagacagcgactcagactcagatagcgactcagattcagatagcgattcagactcagacagcgactcagattcagatagcgattcggactcagacagcgattcagattcagacagcgactcagactcggatagcgattcagattcagatagcgattcggattcagacagtgattcagattcagacagcgactcagactcggatagcgactcagactcagacagcgattcagactcagatagcgactcagactcggatagcgactcggattcagatagcgactcagactcagatagtgactccgattcaagagttacaccaccaaataatgaacagaaagcaccatcaaatcctaaaggtgaagtaaaccattctaataaggtatcaaaacaacacaaaactgatgctttaccagaaacaggagataagagcgaaaacacaaatgcaactttatttggtgcaatgatggcattattaggatcattactattgtttagaaaacgcaagcaagatcataaagaaaaagcgtaa

SEQ ID NO: 14 - protein, 1018 aa, Staphylococcus aureus
MKNNLRYGIRKHKLGAASVFLGTMIVVGMGQDKEAAASEQKTTTVEENGNSATDNKTSETQTTATNVNHIEETQSYNATVTEQPSNATQVTTEEAPKAVQAPQTAQPANIETVKEEVVKEEAKPQVKETTQSQDNSGDQRQVDLTPKKATQNQVAETQVEVAQPRTASESKPRVTRSADVAEAKEASNAKVETGTDVTSKVTVEIGSIEGHNNTNKVEPHAGQRAVLKYKLKFENGLHQGDYFDFTLSNNVNTHGVSTARKVPEIKNGSVVMATGEVLEGGKIRYTFTNDIEDKVDVTAELEINLFIDPKTVQTNGNQTITSTLNEEQTSKELDVKYKDGIGNYYANLNGSIETFNKANNRFSHVAFIKPNNGKTTSVTVTGTLMKGSNQNGNQPKVRIFEYLGNNEDIAKSVYANTTDTSKFKEVTSNMSGNLNLQNNGSYSLNIENLDKTYVVHYDGEYLNGTDEVDFRTQMVGHPEQLYKYYYDRGYTLTWDNGLVLYSNKANGNEKNGPIIQNNKFEYKEDTIKETLTGQYDKNLVTTVEEEYDSSTLDIDYHTAIDGGGGYVDGYIETIEETDSSAIDIDYHTAVDSEAGHVGGYTESSEESNPIDFEESTHENSKHHADVVEYEEDTNPGGGQVTTESNLVEFDEESTKGIVTGAVSDHTTVEDTKEYTTESNLIELVDELPEEHGQAQGPVEEITKNNHHISHSGLGTENGHGNYDVIEEIEENSHVDIKSELGYEGGQNSGNQSFEEDTEEDKPKYEQGGNIVDIDFDSVPQIHGQNKGNQSFEEDTEKDKPKYEHGGNIIDIDFDSVPHIHGFNKHTEIIEEDTNKDKPSYQFGGHNSVDFEEDTLPKVSGQNEGQQTIEEDTTPPIVPPTPPTPEVPSEPETPTPPTPEVPSEPETPTPPTPEVPSEPETPTPPTPEVPAEPGKPVPPAKEEPKKPSKPVEQGKVVTPVIEINEKVKAVAPTKKPQSKKSELPETGGEESTNKGMLFGGLFSILGLALLRRNKKNHKA

SEQ ID NO: 15 - DNA, 2973 bp, Staphylococcus aureus
atgggacaagacaaagaagctgcagcatcagaacaaaagacaactacagtagaagaaaatgggaattcagctactgataataaaacaagtgaaacacaaacaactgcaactaacgttaatcatatagaagaaactcaatcatataacgcaacagtaacagaacaaccgtcaaacgcaacacaagtaacaactgaagaagcaccaaaagcagtacaagcaccacaaactgcacaaccagcaaatatagaaacagttaaagaagaggtagttaaggaagaagcgaaacctcaagttaaggaaacaacacaatctcaagacaatagcggagatcaaagacaagtagatttaacacctaaaaaggctacacaaaatcaagtcgcagaaacacaagttgaagtggcacagccaagaacggcatcagaaagtaagccacgtgtgacaagatcagcagatgtagcggaagctaaggaagctagtaacgcgaaagtggaaacgggtacagatgtaacaagtaaagttacagtagaaattggttctattgaggggcataacaatacaaataaagtagaacctcatgcaggacaacgagcggtactaaaatataagttgaaatttgagaatggtttacatcaaggtgactactttgactttactttatcaaataatgtaaatacgcatggcgtatcaactgctagaaaagtaccagaaattaaaaatggttcagtcgtaatggcgacaggtgaagttttagaaggtggaaagattagatatacatttacaaatgatattgaagataaggttgatgtaacggctgaactagaaattaatttatttattgatcctaaaactgtacaaactaatggaaatcaaactataacttcaacactaaatgaagaacaaacttcaaaggaattagatgttaaatataaagatggtattgggaattattatgccaatttaaatggatcgattgagacatttaataaagcgaataatagattttcgcatgttgcatttattaaacctaataatggtaaaacgacaagtgtgactgttactggaactttaatgaaaggtagtaatcagaatggaaatcaaccaaaagttaggatatttgaatacttgggtaataatgaagacatagcgaagagtgtatatgcaaatacgacagatacttctaaatttaaagaagtcacaagtaatatgagtgggaatttgaatttacaaaataatggaagctattcattgaatatagaaaatctagataaaacttatgttgttcactatgatggagagtatttaaatggtactgatgaagttgattttagaacacaaatggtaggacatccagagcaactttataagtattattatgatagaggatataccttaacttgggataatggtttagttttatacagtaataaagcgaacggaaatgagaaaaatggtccgattattcaaaataataaatttgaatataaagaagatacaattaaagaaactcttacaggtcaatatgataagaatttagtaactactgttgaagaggaatatgattcatcaactcttgacattgattaccacacagctatagatggtggaggtggatatgttgatggatacattgaaacaatagaagaaacggattcatcagctattgatatcgattaccatactgctgtggatagcgaagcaggtcacgttggaggatacactgagtcctctgaggaatcaaatccaattgactttgaagaatctacacatgaaaattcaaaacatcacgctgatgttgttgaatatgaagaagatacaaacccaggtggtggtcaggttactactgagtctaacttagttgaatttgacgaagagtctacaaaaggtattgtaactggcgcagtgagcgatcatacaacagttgaagatacgaaagaatatacaactgaaagtaatctgattgaattagtggatgaattacctgaagagcatggtcaagcacaaggaccagtcgaggaaattactaaaaacaatcatcatatttctcattctggtttaggaactgaaaatggtcacgggaattatgacgtgattgaagaaatcgaagaaaatagccacgttgatattaagagtgaattaggttatgaaggtggccaaaatagcggtaaccagtcattcgaggaagacacagaagaagacaaacctaaatatgaacaaggtggcaatatcgtagatatcgattttgatagtgtacctcaaattcatggtcaaaataaaggtaatcagtcattcgaggaagatacagaaaaagacaaacctaagtatgaacatggcggtaacatcattgatatcgacttcgacagtgtgccacatattcacggattcaataagcacactgaaattattgaagaagatacaaataaagataaaccaagttatcaattcggtggacacaatagtgttgactttgaagaagatacacttccaaaagtaagcggccaaaatgaaggtcaacaaacgattgaagaagatacaacacctccaatcgtgccaccaacgccaccgacaccagaagtaccaagtgagccggaaacaccaacgccaccaacaccagaagtaccaagtgagccggaaacaccaacaccaccgacaccagaagtgccgagtgagccagaaactccaacaccgccaacaccagaggtaccagctgaacctggtaaaccagtaccacctgccaaagaagaacctaaaaagccttctaaaccagtggaacaaggtaaagtagtaacacctgttattgaaatcaatgaaaaggttaaagcagtggcaccaactaaaaaaccacaatctaagaaatctgaactacctgaaacaggtggagaagaatcaacaaacaaaggtatgttgttcggcggattattcagcattctaggtttagcattattacgcagaaataaaaagaatcacaaagcataa

SEQ ID NO: 16 - protein, 116 aa, Staphylococcus aureus
MKIRKSILAGTLAIVLASPLVTNLDKNEAQASTSLPTSNEYQNEKLANELKSLLDELNVNELATGSLNTYYKRTIKISGLKAMYALKSKDFKKMSEAKYQLQKIYNEIDEALKSKY

SEQ ID NO: 17 - DNA, 351 bp, Staphylococcus aureus
atgaaaattagaaaatctatacttgcgggaactttagcaatcgttttagcatcaccactagtaactaatctagataaaaatgaggcacaagctagcacaagcttgccaacatcgaatgaatatcaaaacgaaaagttagctaatgaattaaaatcgttattagatgaactaaatgttaatgaattagctactggaagtttaaacacttattataagcgaactataaaaatttcaggtctaaaagcaatgtatgctcttaagtcaaaagactttaagaaaatgtcagaagcaaaatatcaacttcaaaagatttataacgaaattgacgaagcactaaaaagtaaatattaa

SEQ ID NO: 18 - protein, 149 aa, Staphylococcus aureus
MKKKLATTVLALSFLTAGISTHHHSAKAFTFEPFPTNEEIESNKKLLEKEKAYKESFKNSGLPTTLGKLDERLRNYLKKGTKNSAQFEKMVILTENKGYYTVYLNTPLAEDRKNVELLGKMYKTYFFKKGESKSSYVINGPGKTNEYAY

SEQ ID NO: 19 - DNA, 450 bp, Staphylococcus aureus
atgaaaaagaaattagcaacaacagttttagcattaagttttttaacggcaggaatcagtacacaccatcattcagcgaaagcttttacttttgaaccgtttcctacaaatgaagaaatagaatcaaataagaaattgttagagaaagaaaaagcttataaagaatcatttaaaaatagtggtcttcctacaacactaggaaaattagatgaacgtttgagaaattatttaaagaaaggcacaaaaaattctgctcaatttgaaaaaatggttattttaactgaaaataaaggttactatacagtatatctgaatacaccacttgctgaagatagaaaaaatgttgagttactaggtaaaatgtataaaacatacttctttaaaaaaggagagtctaaatcatcttatgtaattaatggtcctggaaaaactaatgaatatgcatactaa

SEQ ID NO: 20 - protein, 319 aa, Staphylococcus aureus
MKTRIVSSVTTTLLLGSILMNPVANAADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 21 - DNA, 960 bp, Staphylococcus aureus
atgaaaacacgtatagtcagctcagtaacaacaacactattgctaggttccatattaatgaatcctgtcgctaatgccgcagattctgatattaatattaaaaccggtactacagatattggaagcaatactacagtaaaaacaggtgatttagtcacttatgataaagaaaatggcatgcacaaaaaagtattttatagttttatcgatgataaaaatcataataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggctctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcactggacttcaacaaattggaaaggtaccaatactaaagataaatggatagatcgttcttcagaaagatataaaatcgattgggaaaaagaagaaatgacaaattaa

SEQ ID NO: 22 - protein, 165 aa, Staphylococcus aureus
MKNKLIAKSLLTLAAIGITTTTIASTADASEGYGPREKKPVSINHNIVEYNDGTFKYQSRPKFNSTPKYIKFKHDYNILEFNDGTFEYGARPQFNKPAAKTDATIKKEQKLIQAQNLVREFEKTHTVSAHRKAQKAVNLVSFEYKVKKMVLQERIDNVLKQGLVK

SEQ ID NO: 23 - DNA, 498 bp, Staphylococcus aureus
atgaaaaataaattgatagcaaaatctttattaacattagcggcaataggtattactacaactacaattgcgtcaacagcagatgcgagcgaaggatacggtccaagagaaaagaaaccagtgagtattaatcacaatatcgtagagtacaatgatggtacttttaaatatcaatctagaccaaaatttaactcaacacctaaatatattaaattcaaacatgactataatattttagaatttaacgatggtacattcgaatatggtgcacgtccacaatttaataaaccagcagcgaaaactgatgcaactattaaaaaagaacaaaaattgattcaagctcaaaatcttgtgagagaatttgaaaaaacacatactgtcagtgcacacagaaaagcacaaaaggcagtcaacttagtttcgtttgaatacaaagtgaagaaaatggtcttacaagagcgaattgataatgtattaaaacaaggattagttaaataa

SEQ ID NO: 24 - protein, 15 aa, Artificial Sequence
  AviTag Sequence
GLNDIFEAQKIEWHE

SEQ ID NO: 25 - DNA, 45 bp, Artificial Sequence
  DNA encoding AviTag sequence of SEQ ID NO 24
ggcctgaatgacatctttgaagcacagaaaatcgaatggcacgaa

(19) United States
(12) Patent Application Publication
(10) Pub. No.: US 2017/0023569 A1
(43) Pub. Date: Jan. 26, 2017

(54) DIAGNOSTIC DEVICE AND METHOD FOR DETECTION OF STAPHYLOCOCCUS INFECTION

(71) Applicants: John L. DAISS, Rochester, NY (US); Kohei NISHITANI, Rochester, NY (US); Edward M. SCHWARZ, Rochester, NY (US); Stephen L. KATES, Pittsford, NY (US)

(72) Inventors: John L. DAISS, Rochester, NY (US); Kohei NISHITANI, Rochester, NY (US); Edward M. SCHWARZ, Rochester, NY (US); Stephen L. KATES, Pittsford, NY (US)

(73) Assignee: UNIVERSITY OF ROCHESTER, Rochester, NY (US)

(21) Appl. No.: 15/110,656

(22) PCT Filed: Jan. 12, 2015
(86) PCT No.: PCT/US15/11068

§ 371 Date: Jul. 8, 2016

Related U.S. Application Data
(60) Provisional application No. 61/926,065, filed on Jan. 10, 2014.

(51) Int. Cl. G01N 33/569

(52) U.S. Cl. CPC G01N 33/56938; G01N 2469/20

(57) ABSTRACT

Disclosed herein are diagnostic devices, kits, and methods for the detection of an active Staphylococcus infection in an individual. Utilizing a sample from the individual, antibodies specific for one or more Staphylococcus polypeptides are detected, where the detection of a threshold number of antibodies specific for one or more Staphylococcus polypeptides indicates the presence of an active Staphylococcus infection. Exemplary panels of Staphylococcus polypeptides that can be used with a high degree of specificity and sensitivity are disclosed.

This application claims the priority benefit of U.S. Provisional Patent Application Ser. No. 61/926,065 filed Jan. 10, 2014, which is hereby incorporated by reference in its entirety.

### FIELD OF USE

Disclosed herein are methods and diagnostic devices for the detection of an active Staphylococcus infection.

### BACKGROUND

Infection is one of the most serious complications after orthopaedic surgery, occurring in 0.4˜3.0% of primary or revision total joint arthroplasty (“TJA”), 0.5˜2% of closed fractures, and approximately 30% of open fractures (Cram P, et al. JAMA 308, 1227-36 (2012); Schenker M L, et al. J Bone Joint Surg Am. 94,1057-64 (2012)). Staphylococci accounts for ˜80% of these infections, of which ˜50% are caused by methicillin-resistant S. aureus (“MRSA”) (Cram P, et al. JAMA 308, 1227-36 (2012); Schenker M L, et al. J Bone Joint Surg Am. 94, 1057-64 (2012)). A major challenge in caring for patients with S. aureus infections is that many are culture negative despite clinical signs and symptoms, which often delays appropriate early antibiotic therapy.

Limitations of serum-based diagnostics for orthopaedic S. aureus infections have been recently published (Gedbjerg N, et al., J. Bon. Joint Surg. 95:e171(1-9) (2013)). In particular, it has been observed that patients with ongoing S. aureus infections have surprisingly modest increases in antibody levels for specific S. aureus antigens and that the predictive power using a single antigen is too low to have clinical impact (about 70% sensitivity and specificity).

Thus, there remains a great need for a rapid, sensitive, inexpensive and non-invasive diagnostic test for the early identification of infected patients.

The present invention is directed to overcoming these and other deficiencies in the art.

### SUMMARY OF THE DISCLOSURE

A first aspect relates to a diagnostic device that includes: a substrate comprising a plurality of discrete sites and one of a plurality of polypeptides present at each of the plurality of discrete sites, each of the polypeptides comprising an epitope that binds specifically to an antibody present in serum of an individual having an active Staphylococcus infection, wherein, upon exposure to the sample of an individual, the specific binding of a threshold number of the plurality of polypeptides to antibodies in the serum indicates the presence of an active Staphylococcus infection.

A second aspect relates to a diagnostic device that includes a substrate comprising an Iron-regulated surface determinant protein B (IsdB) polypeptide present on the substrate, wherein the IsdB polypeptide comprises an epitope that binds specifically to an antibody present in serum of an individual having an active Staphylococcus infection, wherein, upon exposure to the serum of an individual, the specific binding of the IsdB polypeptide to antibodies in the serum indicates the presence of an active Staphylococcus infection. In certain embodiments, the diagnostic device consists of the IsdB polypeptide.

A third aspect relates to a kit containing a diagnostic device according to the first or second aspects disclosed herein.

A fourth aspect relates to a method of detecting an active Staphylococcus infection in an individual. This method includes obtaining a sample from an individual; exposing the obtained sample to a plurality of polypeptides bound to a surface, each of the polypeptides comprising an epitope that binds specifically to an antibody present in serum of an individual having an active Staphylococcus infection; and determining whether, after said exposing, the specific binding of a threshold number of the plurality of polypeptides to antibodies in the sample occurred, thereby indicating the presence of an active Staphylococcus infection.

A fifth aspect relates to a method of detecting an active Staphylococcus infection in an individual. This method includes obtaining a sample from an individual; exposing the obtained sample to an IsdB polypeptide present on a surface, the IsdB polypeptide comprising an epitope that binds specifically to an antibody present in serum of an individual having an active Staphylococcus infection; and determining whether, after said exposing, the specific binding of the IsdB polypeptide to antibodies in the sample occurred, thereby indicating the presence of an active Staphylococcus infection.

A sixth aspect relates to a method of identifying a joint replacement patient having a higher likelihood of needing revision joint replacement surgery. This method includes performing the method according to the fourth aspect disclosed herein to identify a patient that received a total joint replacement and has an active Staphylococcus infection; and determining whether the level of anti-Amd, anti-Gmd, or anti-ClfB antibodies, as measured during said performing, is lower than a threshold titer; wherein an anti-Amd titer, an anti-Gmd titer, an anti-ClfB titer, or any combination thereof, that is lower than a threshold titer level indicates that the patient is likely to need revision joint replacement surgery.

As demonstrated in the accompanying examples, the inventors have measured the humoral immune response against S. aureus, and tested the hypothesis that patients with deep musculoskeletal S. aureus infections have high levels of circulating antibodies against selected bacterial surface and secreted proteins. The results of this analysis demonstrate the achievement of a panel of markers that, together, allow for high specificity and sensitivity in the detection of active S. aureus infection in a rapid, non-invasive diagnostic assay format. This diagnostic is significantly superior versus the current standard of care. Using this diagnostic, it is also possible to identify total joint replacement patients that have an active Staphylococcus infection and, based on certain antibody titers, are likely to require a revision total joint replacement.

### BRIEF DESCRIPTION OF DRAWINGS

FIG. 1A illustrates antibody levels of Control and Patient sera against 12 antigens (Gmd, Amd, IsdA, IsdB, IsdH, ClfA, ClfB, FnbpA, CHIPS, SCIN, Hla and Efb) shown by dot plot together with the median and interquartile. Each value is expressed as a ratio to the median of Control sera (*mean p<0.05; ** mean p<0.01 versus Control with Mann-Whitney U test). FIG. 1B is an ROC curve of IsdB (left panel), which shows highest AUC (0.80) among the 12 antigens. The cutoff value is defined as the closest point from top left corner to ROC curve and indicated in right panel.

FIG. 2 illustrates the improved sensitivity and specificity in using a combination of antigens. Use of 8 antigens (Gmd, Amd, IsdA, IsdB, IsdH, ClfA, ClfB, FnbpA) for detection of corresponding antibodies showed greater AUC (0.83) than any single antibody in left panel. A cutoff value of 1.49 was defined as the closest point from top left corner to ROC curve and indicated in right panel, and cutoff value of 2.04 was defined as the maximum of Control, both are indicated in right panel.

FIG. 3 illustrates the improved sensitivity and specificity in using a different combination of antigens. Use of all 12 antigen for detection of corresponding antibodies showed greater AUC (0.87) than that shown in FIG. 2. A cutoff value of 4.42 (condition) achieved diagnostic power of 77.1% sensitivity, 80.0% specificity and 77.1% PPV), whereas a more stringent cutoff value of 5.99 (condition) identified 60% of the infected patients with no false positives.

### DETAILED DESCRIPTION OF INVENTION

The present invention relates to diagnostic devices and methods for use in the detection of active Staphylococcus infections. It is contemplated herein that the diagnostic devices and methods of the present invention can be used to detect the presence of active infections against one or more of S. aureus, S. epidermidis, S. lugdunensis, S. saprophyticus, S. haemolyticus, S. caprae, and S. simiae. In certain embodiments, the present invention can discriminate between one or more of these Staphylococcus species.

In the various embodiments, at least one polypeptide or a plurality of polypeptides is bound to or present on a surface of the diagnostic device. Where a plurality of polypeptides is used, they are preferably bound to the surface at discrete locations. Each of the polypeptides includes an epitope that binds (or multiple epitopes that bind) specifically to an antibody present in a sample from an individual having an active Staphylococcus infection. As a consequence, antibody binding events for each of the polypeptides can be monitored and detected, allowing for an assessment of whether one or more antibodies are present in a particular sample.

A biological sample can be obtained and/or derived from, for example, blood, plasma, serum, homogenates of tissues, synovial fluid, saliva, sputum, amniotic fluid, cerebrospinal fluid, peritoneal fluid, lung lavage fluid, semen, lymphatic fluid, tears, or prostatic fluid. These samples can be obtained using standard procedures. Preferred samples are those fluids that are abundant in IgG antibodies.

These samples are obtained from an individual suspected of possessing a Streptococcus infection on the basis of clinical signs and symptoms of such infection being present. These clinical signs and symptoms include, without limitation, localized pain which is frequently exacerbated by motion, local warmth, tenderness, edema, erythema, drainage, and effusion. Patients that are at-risk of infection (and who should be regularly monitored) include those who have a received an orthopedic implant of one form or another, including without limitation, a joint prosthesis, a graft or synthetic implant, and those who undergo a surgical procedure involving joint infiltration or disruption of bone surfaces.

The diagnostic device can include any of a variety of formats, including, without limitation, multi-well ELISA plates, multiple distinct beads, and arrays formed on glass slides, silicon, or any other substrates suitable for labeled or label-free detection. These are described in greater detail below.

Regardless of the format of the device, polypeptides used for the capture of circulating antibodies (in samples from an individual) can be produced in purified form and then used to fabricate the diagnostic device. The polypeptides can be a full-length protein (e.g., a mature protein), a polypeptide fragment of the full-length protein that includes an antigenic region of interest, or a fusion protein that includes the full-length protein or the polypeptide fragment thereof along with one or more additional amino acids or amino acid sequences that aid in purification and/or fabrication of the device. An antigenic region of interest is a portion of a full-length protein that contains a polypeptide sequence containing a linear or conformational epitope, and which is capable of either inducing an antibody response (upon administration) or binding specifically to an antibody raised against a full-length protein that contains the antigenic region of interest.

Amino acid sequences that aid in purification include, without limitation, any of a variety of well-known affinity purification sequences such as chitin binding protein (CBP), maltose binding protein (MBP), glutathione-S-transferase (GST), myc tag, HA tag, Flag-peptide, KT3 epitope, alpha-tubulin epitope, T7 gene 10 protein peptide tag, strep-tag, bovine pancreatic trypsin inhibitor (BPTI), polyhistidine tag (6× His), a polyarginine tag, S-tag, thioredoxin, staphylococcal protein A tag, AviTag epitope, a biotin tag, a TAP-tag, an SBP-tag, a calmodulin-binding peptide tag, a cellulose-binding domain tag, a DsbA tag, and a NusA tag.

Amino acids or amino acid sequences that aid in device fabrication include, without limitation, biotin (avidin or streptavidin), Protein A/G, and amino acids modified with e.g., NHS Ester, Azlactone, Aldehyde, Carbonyl diimidazole, maleimide, iodoacetyl, pyridyl disulfide, hydrazide, and EDC or DCC carbodiimide. Of course, other attachment chemistries can also be utilized.

The polypeptides can be recovered from Staphylococcus samples grown in vitro, the polypeptides can be synthesized using solid phase synthesis procedures, or the polypeptides can recombinantly produced. Regardless of how the polypeptides are produced, they are preferably isolated and purified prior to their use in fabricating the diagnostic device.

Any of a variety of secreted or surface-exposed Staphylococcus antigen can be used in forming the diagnostic device of the present invention. Exemplary Staphylococcus antigen include, without limitation, Glucosaminidase (Gmd), Amidase (Amd), Iron-regulated surface determinant protein A (IsdA), Iron-regulated surface determinant protein B (IsdB), Iron-regulated surface determinant protein H (IsdH), Clumping Factor A (ClfA), Clumping Factor B (ClfB), Fibronectin Binding Protein A (FnbpA), Staphylococcus Complement Inhibitor (SCIN), Chemotaxis Inhibitory Protein of Staphylococcus aureus (CHIPS), α-Hemolysin (Hla), and Extracellular Fibrinogen-binding Protein (Efb). Each of these exemplary polypeptides is described briefly in the paragraphs below.

The AtlA enzyme is comprised of an N-acetylmuramoyl-L-alanine-amidase (Amd) (62kD) and endo-β-N-acetylglucosaminidase (Gmd) (53kD), which are produced from the same AtlA precursor protein via a cleavage process (Baba and Schneewind, “Targeting of Muralytic Enzymes to the Cell Division Site of Gram-Positive Bacteria: Repeat Domains Direct Autolysin to the Equatorial Surface Ring of Staphylococcus aureus,” EMBO J. 17(16):4639-46 (1998); Komatsuzawa et al., “Subcellular Localization of the Major Autolysin, ATL and Its Processed Proteins in Staphylococcus aureus,” Microbiol Immunol. 41:469-79 (1997); Oshida et al., “A Staphylococcus aureus Autolysin That Has an N-acetylmuramoyl-L-alanine Amidase Domain and an Endo-beta-N-acetylglucosaminidase Domain: Cloning, Sequence Analysis, and Characterization,” Proc. Nat'l. Acad. Sci. U.S.A. 92(1):285-9 (1995), which are hereby incorporated by reference in their entirety).

Gmd contains the amino acid sequence shown below (SEQ ID NO: 1).

| | |
|---|---|
| 1 | AYTVTKPQTT QTVSKIAQVK PNNTGIRASV YEKTAKNGAK YADRTFYVTK ERAHGNETYV |
| 61 | LLNNTSHNIP LGWFNVKDLN VQNLGKEVKT TQKYTVNKSN NGLSMVPWGT KNQVILTGNN |
| 121 | IAQGTFNATK QVSVGKDVYL YGTINNRTGW VNAKDLTAPT AVKPTTSAAK DYNYTYVIKN |
| 181 | GNGYYYVTPN SDTAKYSLKA FNEQPFAVVK EQVINGQTWY YGKLSNGKLA WIKSTDLAKE |
| 241 | LIKYNQTGMT LNQVAQIQAG LQYKPQVQRV PGKWTDANFN DVKHAMDTKR LAQDPALKYQ |
| 301 | FLRLDQPQNI SIDKINQFLK GKGVLENQGA AFNKAAQMYG INEVYLISHA LLETGNGTSQ |
| 361 | LAKGADVVNN KVVTNSNTKY HNVFGIAAYD NDPLREGIKY AKQAGWDTVS KAIVGGAKFI |
| 421 | GNSYVKAGQN TLYKMRWNPA HPGTHQYATD VDWANINAKI IKGYYDKIGE VGKYFDIPQY |

Residues 8-144 (italics) represent the R3 domain, and the remaining C-terminal residues correspond to the catalytic glucosaminidase domain. The sequence above corresponds to residues 768 to 1247 of the autolysin amino acid sequence reported at Genbank Accession YP_493653, which is hereby incorporated by reference in its entirety. The nucleotide sequence encoding the above-identified Gmd is provided at Genbank Accession NC_007793, which is hereby incorporated by reference in its entirety, and set forth below (SEQ ID NO: 2).

atggcgaaaaaattcaattacaaactaccatcaatggttgcattaacgct
tgtaggttcagcagtcactgcacatcaagttcaagcagctgagacgacac
aagatcaaactactaataaaaacgttttagatagtaataaagttaaagca
actactgaacaagcaaaagctgaggtaaaaaatccaacgcaaaacatttc
tggcactcaagtatatcaagaccctgctattgtccaaccaaaaacagcaa
ataacaaaacaggcaatgctcaagtaagtcaaaaagttgatactgcacaa
gtaaatggtgacactcgtgctaatcaatcagcgactacaaataatacgca
gcctgttgcaaagtcaacaagcactacagcacctaaaactaacactaatg
ttacaaatgctggttatagtttagttgatgatgaagatgataattcagaa
aatcaaattaatccagaattaattaaatcagctgctaaacctgcagctct
tgaaacgcaatataaaaccgcagcacctaaagctgcaactacatcagcac
ctaaagctaaaactgaagcgacacctaaagtaactacttttagcgcttca
gcacaaccaagatcagttgctgcaacaccaaaaacgagtttgccaaaata
taaaccacaagtaaactcttcaattaacgattacattcgtaaaaataact
taaaagcacctaaaattgaagaagattatacatcttacttccctaaatac
gcataccgtaacggcgtaggtcgtcctgaaggtatcgtagttcatgatac
agctaatgatcgttcgacgataaatggtgaaattagttatatgaaaaata
actatcaaaacgcattcgtacatgcatttgttgatggggatcgtataatc
gaaacagcaccaacggattacttatcttggggtgtcggtgcagtcggtaa
ccctagattcatcaatgttgaaatcgtacacacacacgactatgcttcat
ttgcacgttcaatgaataactatgctgactatgcagctacacaattacaa
tattatggtttaaaaccagacagtgctgagtatgatggaaatggtacagt
atggactcactacgctgtaagtaaatatttaggtggtactgaccatgccg
atccacatggatatttaagaagtcataattatagttatgatcaattatat
gacttaattaatgaaaaatatttaataaaaatgggtaaagtggcgccatg
gggtacgcaatctacaactacccctactacaccatcaaaaccaacaacac
cgtcgaaaccatcaactggtaaattaacagttgctgcaaacaatggtgtc
gcacaaatcaaaccaacaaatagtggtttatatactactgtatacgacaa
aactggtaaagcaactaatgaagttcaaaaaacatttgctgtatctaaaa
cagctacattaggtaatcaaaaattctatcttgttcaagattacaattct
ggtaataaatttggttgggttaaagaaggcgatgtggtttacaacacagc
taaatcacctgtaaatgtaaatcaatcatattcaatcaaacctggtacga
aactttatacagtaccttggggtacatctaaacaagttgctggtagtgtg
tctggctctggaaaccaaacatttaaggcttcaaagcaacaacaaattga
taaatcaatttatttatatggctctgtgaatggtaaatctggttgggtaa
gtaaagcatatttagttgatactgctaaacctacgcctacaccaacacct
aagccatcaacacctacaacaaataataaattaacagtttcatcattaaa
cggtgttgctcaaattaatgctaaaaacaatggcttattcactacagttt
atgacaaaactggtaagccaacgaaagaagttcaaaaaacatttgctgta
acaaaagaagcaagtttaggtggaaacaaattctacttagttaaagatta
caatagtccaactttaattggttgggttaaacaaggtgacgttatttata
acaatgcaaaatcacctgtaaatgtaatgcaaacatatacagtaaaacca
ggcactaaattatattcagtaccttggggcacttataaacaagaagctgg
tgcagtttctggtacaggtaaccaaacttttaaagcgactaagcaacaac
aaattgataaatctatctatttatttggaactgtaaatggtaaatctggt
tgggtaagtaaagcatatttagctgtacctgctgcacctaaaaaagcagt
agcacaaccaaaaacagctgtaaaagcttatactgttactaaaccacaaa
cgactcaaacagttagcaagattgctcaagttaaaccaaacaacactggt
attcgtgcttctgtttatgaaaaaacagcgaaaaacggtgcgaaatatgc
agaccgtacgttctatgtaacaaaagagcgtgctcatggtaatgaaacgt
atgtattattaaacaatacaagccataacatcccattaggttggttcaat
gtaaaagacttaaatgttcaaaacttaggcaaagaagttaaaacgactca
aaaatatactgttaataaatcaaataacggcttatcaatggttccttggg
gtactaaaaaccaagtcattttaacaggcaataacattgctcaaggtaca
tttaatgcaacgaaacaagtatctgtaggcaaagatgtttatttatacgg
tactattaataaccgcactggttgggtaaatgcaaaagatttaactgcac
caaccgctgtgaaaccaactacatcagctgccaaagattataactacact
tatgtaattaaaaatggtaatggttattactatgtaacaccaaattctga
tacagctaaatactcattaaaagcatttaatgaacaaccattcgcagttg
ttaaagaacaagtcattaatggacaaacttggtactatggtaaattatct
aacggtaaattagcatggattaaatcaactgatttagctaaagaattaat
taagtataatcaaacaggtatgacattaaaccaagttgctcaaatacaag
ctggtttacaatataaaccacaagtacaacgtgtaccaggtaagtggaca
gatgctaactttaatgatgttaagcatgcaatggatacgaagcgtttagc
tcaagatccagcattaaaatatcaattcttacgcttagaccaaccacaaa
atatttctattgataaaattaatcaattcttaaaaggtaaaggtgtatta
gaaaaccaaggtgctgcatttaacaaagctgctcaaatgtatggcattaa
tgaagtttatcttatctcacatgccctattagaaacaggtaacggtactt
ctcaattagcgaaaggtgcagatgtagtgaacaacaaagttgtaactaac
tcaaacacgaaataccataacgtatttggtattgctgcatatgataacga
tcctttacgtgaaggtattaaatatgctaaacaagctggttgggacacag
tatcaaaagcaatcgttggtggtgctaaattcatcggcaactcatatgta
aaagctggtcaaaatacactttacaaaatgagatggaatcctgcacatcc
aggaacacaccaatatgctacagatgtagattgggctaacatcaatgcta
aaatcatcaaaggctactatgataaaattggcgaagtcggcaaatacttc
gacatcccacaatataaataa

A number of homologous Staphylococcus Gmd amino acid and nucleotide sequences are available from Genbank. Similarly, a number of homologous Gmd amino acid and nucleotide sequences from other Staphylococcus species are also available from Genbank.

Any one or more of these Gmd sequences, whether now known or hereafter identified, can be used in accordance with the present invention. In certain embodiments, the homologous amino acid sequences comprise at least about 80 percent identity, at least about 85 percent identity, at least about 90 percent identity, or at least about 95 percent identity to the full length Gmd amino acid sequence provided above. In other embodiments, the homologous amino acid sequences comprise at least about 80 percent identity, at least about 85 percent identity, at least about 90 percent identity, or at least about 95 percent identity to the R3 domain of the Gmd amino acid sequence provided above or the catalytic domain of the Gmd amino acid sequence provided above.

The use of Gmd polypeptide fragments is also contemplated, including fragments containing either linear or conformational epitopes. Such fragments may include at least 20, at least 30, at least 40, at least 50, at least 60, at least 70, at least 80, at least 90, or at least 100 contiguous amino acids. Exemplary polypeptide fragments include those containing all or part of the R3 domain, as well as those containing all or part of the Gmd catalytic domain.

Amd contains the amino acid sequence shown below (SEQ ID NO: 3):

| | |
|---|---|
| 1 | SASAQPRSVA ATPKTSLPKY KPQVNSSIND YIRKNNLKAP KIEEDYTSYF PKYAYRNGVG |
| 61 | RPEGIVVHDT ANDRSTINGE ISYMKNNYQN AFVHAFVDGD RIIETAPTDY LSWGVGAVGN |
| 121 | PRFINVEIVH THDYASFARS MNNYADYAAT QLQYYGLKPD SAEYDGNGTV WTHYAVSKYL |
| 181 | GGTDHADPHG YLRSHNYSYD QLYDLINEKY LIKMGKVAPW GTQSTTTPTT PSKPTTPSKP |
| 241 | STGKLTVAAN NGVAQIKPTN SGLYTTVYDK TGKATNEVQK TFAVSKTATL GNQKFYLVQD |
| 301 | YNSGNKFGWV KEGDVVYNTA KSPVNVNQSY SIKPGTKLYT VPWGTSKQVA GSVSGSGNQT |
| 361 | FKASKQQQID KSIYLYGSVN GKSGWVSKAY LVDTAKPTPT PTPKPSTPTT NNKLTVSSLN |
| 421 | GVAQINAKNN GLFTTVYDKT GKPTKEVQKT FAVTKEASLG GNKFYLVKDY NSPTLIGWVK |
| 481 | QGDVIYNNAK SPVNVMQTYT VKPGTKLYSV PWGTYKQEAG AVSGTGNQTF KATKQQQIDK |
| 541 | SIYLFGTVNG KSGWVSKAYL AVPAAPKKAV AQPKTAVK |

Residues 1-244 correspond to the catalytic domain, and residues 244-391 and 413-560 represent the R1 and R2 domains, respectively. The sequence above corresponds to residues 198 to 775 of the autolysin amino acid sequence reported at Genbank Accession YP_493653, which is hereby incorporated by reference in its entirety. The nucleotide sequence encoding the above-identified Gmd is provided at Genbank Accession NC_007793, which is hereby incorporated by reference in its entirety, and set forth above for Gmd.

A number of homologous Staphylococcus Amd amino acid and nucleotide sequences are available from Genbank. Similarly, a number of homologous Amd amino acid and nucleotide sequences from other Staphylococcus species are also available from Genbank.

Any one or more of these Amd sequences, whether now known or hereafter identified, can be used in accordance with the present invention. In certain embodiments, the homologous amino acid sequences comprise at least about 80 percent identity, at least about 85 percent identity, at least about 90 percent identity, or at least about 95 percent identity to the full length Amd amino acid sequence provided above. In other embodiments, the homologous amino acid sequences comprise at least about 80 percent identity, at least about 85 percent identity, at least about 90 percent identity, or at least about 95 percent identity to the R1 and/or R2 domain of the Amd amino acid sequence provided above or the catalytic domain of the Amd amino acid sequence provided above.

The use of Amd polypeptide fragments is also contemplated, including fragments containing either linear or conformational epitopes. Such fragments may include at least 20, at least 30, at least 40, at least 50, at least 60, at least 70, at least 80, at least 90, or at least 100 contiguous amino acids. Exemplary polypeptide fragments include those containing all or part of the R1 domain, all or part of the R2 domain, both the R1 and R2 domains, as well as those containing all or part of the Amd catalytic domain.

Iron-regulated surface determinant protein A (IsdA) is involved in adherence of S. aureus to human desquamated nasal epithelial cells and is required for nasal colonization. IsdA also protects S. aureus against the bactericidal protease activity of apolactoferrin in vitro and confers resistance to bovine lactoferricin. In addition, IsdA is shown to promote resistance to hydrogen peroxide and killing by neutrophils.

An exemplary IsdA has the amino acid sequence of the sequence shown below (SEQ ID NO: 4):

Staphylococcus aureus NCTC 8325 (Genbank Accession Q2FZE9, which is hereby incorporated by reference in its entirety)

| | |
|---|---|
| 1 | MTKHYLNSKY QSEQRSSAMK KITMGTASII LGSLVYIGAD SQQVNAATEA TNATNNQSTQ |
| 61 | VSQATSQPIN FQVQKDGSSE KSHMDDYMQH PGKVIKQNNK YYFQTVLNNA SFWKEYKFYN |
| 121 | ANNQELATTV VNDNKKADTR TINVAVEPGY KSLTTKVHIV VPQINYNHRY TTHLEFEKAI |
| 181 | PTLADAAKPN NVKPVQPKPA QPKTPTEQTK PVQPKVEKVK PTVTTTSKVE DNHSTKVVST |
| 241 | DTTKDQTKTQ TAHTVKTAQT AQEQNKVQTP VKDVATAKSE SNNQAVSDNK SQQTNKVTKH |
| 301 | NETPKQASKA KELPKTGLTS VDNFISTVAF ATLALLGSLS LLLFKRKESK |

Amino acids 1-46 (italics) represent a likely signal peptide, and amino acids 317-350 (italics) likely represent a propeptide sequence that is enzymatically cleaved, e.g., by a sortase. The mature extracellular polypeptide constitutes amino acids 47-316. The nucleotide sequence encoding the above-identified IsdA is provided at Genbank Accession NC_007795, which is hereby incorporated by reference in its entirety, and set forth below (SEQ ID NO: 5).

atgacaaaacattatttaaacagtaagtatcaatcagaacaacgttcatc
agctatgaaaaagattacaatgggtacagcatctatcattttaggttccc
ttgtatacataggcgcagacagccaacaagtcaatgcggcaacagaagct
acgaacgcaactaataatcaaagcacacaagtttctcaagcaacatcaca
accaattaatttccaagtgcaaaaagatggctcttcagagaagtcacaca
tggatgactatatgcaacaccctggtaaagtaattaaacaaaataataaa
tattatttccaaaccgtgttaaacaatgcatcattctggaaagaatacaa
attttacaatgcaaacaatcaagaattagcaacaactgttgttaacgata
ataaaaaagcggatactagaacaatcaatgttgcagttgaacctggatat
aagagcttaactactaaagtacatattgtcgtgccacaaattaattacaa
tcatagatatactacgcatttggaatttgaaaaagcaattcctacattag
ctgacgcagcaaaaccaaacaatgttaaaccggttcaaccaaaaccagct
caacctaaaacacctactgagcaaactaaaccagttcaacctaaagttga
aaaagttaaacctactgtaactacaacaagcaaagttgaagacaatcact
ctactaaagttgtaagtactgacacaacaaaagatcaaactaaaacacaa
actgctcatacagttaaaacagcacaaactgctcaagaacaaaataaagt
tcaaacacctgttaaagatgttgcaacagcgaaatctgaaagcaacaatc
aagctgtaagtgataataaatcacaacaaactaacaaagttacaaaacat
aacgaaacgcctaaacaagcatctaaagctaaagaattaccaaaaactgg
tttaacttcagttgataactttattagcacagttgccttcgcaacacttg
cccttttaggttcattatctttattacttttcaaaagaaaagaatctaaa
taa

A number of homologous Staphylococcus IsdA amino acid and nucleotide sequences are available from Genbank. Similarly, a number of homologous IsdA amino acid and nucleotide sequences from other Staphylococcus species are also available from Genbank.

Any one or more of these IsdA sequences, whether now known or hereafter identified, can be used in accordance with the present invention. In certain embodiments, the homologous amino acid sequences comprise at least about 80 percent identity, at least about 85 percent identity, at least about 90 percent identity, or at least about 95 percent identity to the full length IsdA amino acid sequence provided above. In other embodiments, the homologous amino acid sequences comprise at least about 80 percent identity, at least about 85 percent identity, at least about 90 percent identity, or at least about 95 percent identity to residues 47-316 of the IsdA amino acid sequence provided above.

The use of IsdA polypeptide fragments is also contemplated, including fragments containing either linear or conformational epitopes. Such fragments may include at least 20, at least 30, at least 40, at least 50, at least 60, at least 70, at least 80, at least 90, or at least 100 contiguous amino acids.

IsdB is believed to function as the primary receptor for hemoglobin since its inactivation inhibits the ability of S. aureus to bind hemoglobin. IsdB Binds hemoglobin in a dose-dependent way, and is required for S. aureus growth using hemoglobin as the sole iron source. IsdB is also required for virulence. Like IsdA, IsdB is believed to promote resistance to hydrogen peroxide and killing by neutrophils.

An exemplary IsdB has the amino acid sequence of the sequence shown below (SEQ ID NO: 6):

Staphylococcus aureus NCTC 8325 (Genbank Accession Q2FZF0, which is hereby incorporated by reference in its entirety)

| | |
|---|---|
| 1 | MNKQQKEFKS FYSIRKSSLG VASVAISTLL LLMSNGEAQA AAEETGGTNT EAQPKTEAVA |
| 61 | SPTTTSEKAP ETKPVANAVS VSNKEVEAPT SETKEAKEVK EVKAPKETKE VKPAAKATNN |
| 121 | TYPILNQELR EAIKNPAIKD KDHSAPNSRP IDFEMKKKDG TQQFYHYASS VKPARVIFTD |
| 181 | SKPEIELGLQ SGQFWRKFEV YEGDKKLPIK LVSYDTVKDY AYIRFSVSNG TKAVKIVSST |
| 241 | HFNNKEEKYD YTLMEFAQPI YNSADKFKTE EDYKAEKLLA PYKKAKTLER QVYELNKIQD |
| 301 | KLPEKLKAEY KKKLEDTKKA LDEQVKSAIT EFQNVQPTNE KMTDLQDTKY VVYESVENNE |
| 361 | SMMDTFVKHP IKTGMLNGKK YMVMETTNDD YWKDFMVEGQ RVRTISKDAK NNTRTIIFPY |
| 421 | VEGKTLYDAI VKVHVKTIDY DGQYHVRIVD KEAFTKANTD KSNKKEQQDN SAKKEATPAT |
| 481 | PSKPTPSPVE KESQKQDSQK DDNKQLPSVE KENDASSESG KDKTPATKPT KGEVESSSTT |
| 541 | PTKVVSTTQN VAKPTTASSK TTKDVVQTSA GSSEAKDSAP LQKANIKNTN DGHTQSQNNK |
| 601 | NTQENKAKSL PQTGEESNKD MTLPLMALLA LSSIVAFVLP RKRKN |

Amino acids 1-40 (italics) represent a likely signal peptide, and amino acids 614-645 (italics) likely represent a propeptide sequence that is enzymatically cleaved, e.g., by a sortase. The mature extracellular polypeptide constitutes amino acids 41-613. The nucleotide sequence encoding the above-identified IsdB is provided at Genbank Accession NC_007795, which is hereby incorporated by reference in its entirety, and set forth below (SEQ ID NO: 7).

atgaacaaacagcaaaaagaatttaaatcattttattcaattagaaagtc
atcactaggcgttgcatctgtagcaattagtacacttttattattaatgt
caaatggcgaagcacaagcagcagctgaagaaacaggtggtacaaataca
gaagcacaaccaaaaactgaagcagttgcaagtccaacaacaacatctga
aaaagctccagaaactaaaccagtagctaatgctgtctcagtatctaata
aagaagttgaggcccctacttctgaaacaaaagaagctaaagaagttaaa
gaagttaaagcccctaaggaaacaaaagaagttaaaccagcagcaaaagc
cactaacaatacatatcctattttgaatcaggaacttagagaagcgatta
aaaaccctgcaataaaagacaaagatcatagcgcaccaaactctcgtcca
attgattttgaaatgaaaaagaaagatggaactcaacagttttatcatta
tgcaagttctgttaaacctgctagagttattttcactgattcaaaaccag
aaattgaattaggattacaatcaggtcaattttggagaaaatttgaagtt
tatgaaggtgacaaaaagttgccaattaaattagtatcatacgatactgt
taaagattatgcttacattcgcttctctgtatcaaacggaacaaaagctg
ttaaaattgttagttcaacacacttcaataacaaagaagaaaaatacgat
tacacattaatggaattcgcacaaccaatttataacagtgcagataaatt
caaaactgaagaagattataaagctgaaaaattattagcgccatataaaa
aagcgaaaacactagaaagacaagtttatgaattaaataaaattcaagat
aaacttcctgaaaaattaaaggctgagtacaagaagaaattagaggatac
aaagaaagctttagatgagcaagtgaaatcagctattactgaattccaaa
atgtacaaccaacaaatgaaaaaatgactgatttacaagatacaaaatat
gttgtttatgaaagtgttgagaataacgaatctatgatggatacttttgt
taaacaccctattaaaacaggtatgcttaacggcaaaaaatatatggtca
tggaaactactaatgacgattactggaaagatttcatggttgaaggtcaa
cgtgttagaactataagcaaagatgctaaaaataatactagaacaattat
tttcccatatgttgaaggtaaaactctatatgatgctatcgttaaagttc
acgtaaaaacgattgattatgatggacaataccatgtcagaatcgttgat
aaagaagcatttacaaaagccaataccgataaatctaacaaaaaagaaca
acaagataactcagctaagaaggaagctactccagctacgcctagcaaac
caacaccatcacctgttgaaaaagaatcacaaaaacaagacagccaaaaa
gatgacaataaacaattaccaagtgttgaaaaagaaaatgacgcatctag
tgagtcaggtaaagacaaaacgcctgctacaaaaccaactaaaggtgaag
tagaatcaagtagtacaactccaactaaggtagtatctacgactcaaaat
gttgcaaaaccaacaactgcttcatcaaaaacaacaaaagatgttgttca
aacttcagcaggttctagcgaagcaaaagatagtgctccattacaaaaag
caaacattaaaaacacaaatgatggacacactcaaagccaaaacaataaa
aatacacaagaaaataaagcaaaatcattaccacaaactggtgaagaatc
aaataaagatatgacattaccattaatggcattattagctttaagtagca
tcgttgcattcgtattacctagaaaacgtaaaaactaa

A number of homologous Staphylococcus IsdB amino acid and nucleotide sequences are available from Genbank. Similarly, a number of homologous IsdB amino acid and nucleotide sequences from other Staphylococcus species are also available from Genbank.

Any one or more of these IsdB sequences, whether now known or hereafter identified, can be used in accordance with the present invention. In certain embodiments, the homologous amino acid sequences comprise at least about 80 percent identity, at least about 85 percent identity, at least about 90 percent identity, or at least about 95 percent identity to the full length IsdB amino acid sequence provided above. In other embodiments, the homologous amino acid sequences comprise at least about 80 percent identity, at least about 85 percent identity, at least about 90 percent identity, or at least about 95 percent identity to residues 41-613 of the IsdB amino acid sequence provided above.

The use of IsdB polypeptide fragments is also contemplated, including fragments containing either linear or conformational epitopes. Such fragments may include at least 20, at least 30, at least 40, at least 50, at least 60, at least 70, at least 80, at least 90, or at least 100 contiguous amino acids.

IsdH binds human plasma haptoglobin-hemoglobin complexes, haptoglobin and hemoglobin, although it binds haptoglobin-hemoglobin complexes with significantly higher affinity than haptoglobin alone.

An exemplary IsdH has the amino acid sequence of the sequence shown below (SEQ ID NO: 8):

Staphylococcus aureus NCTC 8325 (Genbank Accession Q2FXJ2, which is hereby incorporated by reference in its entirety)

| | |
|---|---|
| 1 | MNKHHPKLRS FYSIRKSTLG VASVIVSTLF LITSCHQAQA AENTNTSDKI SENQNNNATT |
| 61 | TQPPKDTNQT QPATQPANTA KNYPAADESL KDAIKDPALE NKEHDIGPRE QVNFQLLDKN |
| 121 | NETQYYHFFS IKDPADVYYT KKKAEVELDI NTASTWKKFE VYENNQKLPV RLVSYSPVPE |
| 181 | DHAYIRFPVS DGTQELKIVS STQIDDGEET NYDYTKLVFA KPIYNDPSLV KSDTNDAVVT |
| 241 | NDQSSSVASN QTNTNTSNQN ISTINNANNQ PQATTNMSQP AQPKSSTNAD QASSQPAHET |
| 301 | NSNGNTNDKT NESSNQSDVN QQYPPADESL QDAIKNPAII DKEHTADNWR PIDFQMKNDK |
| 361 | GERQFYHYAS TVEPATVIFT KTGPIIELGL KTASTWKKFE VYEGDKKLPV ELVSYDSDKD |
| 421 | YAYIRFPVSN GTREVKIVSS IEYGENIHED YDYTLMVFAQ PITNNPDDYV DEETYNLQKL |
| 481 | LAPYHKAKTL ERQVYELEKL QEKLPEKYKA EYKKKLDQTR VELADQVKSA VTEFENVTPT |
| 541 | NDQLTDLQEA HFVVFESEEN SESVMDGFVE HPFYTATLNG QKYVVMKTKD DSYWKDLIVE |
| 601 | GKRVTTVSKD PKNNSRTLIF PYIPDKAVYN AIVKVVVANI GYEGQYHVRI INQDINTKDD |
| 661 | DTSQNNTSEP LNVQTGQEGK VADTDVAENS STATNPKDAS DKADVIEPES DVVKDADNNI |
| 721 | DKDVQHDVDH LSDMSDNNHF DKYDLKEMDT QIAKDTDRNV DKDADNSVGM SSNVDTDKDS |
| 781 | NKNKDKVIQL NHIADKNNHT GKAAKLDVVK QNYNNTDKVT DKKTTEHLPS DIHKTVDKTV |
| 841 | KTKEKAGTPS KENKLSQSKM LPKTGETTSS QSWWGLYALL GMLALFIPKF RKESK |

Amino acids 1-40 (italics) represent a likely signal peptide, and amino acids 865-895 (italics) likely represent a propeptide sequence that is enzymatically cleaved, e.g., by a sortase. The mature extracellular polypeptide constitutes amino acids 41-864. The nucleotide sequence encoding the above-identified IsdH is provided at Genbank Accession NC_007795, which is hereby incorporated by reference in its entirety, and set forth below (SEQ ID NO: 9).

atgaacaaacatcacccaaaattaaggtctttctattctattagaaaatc
aactctaggcgttgcatcggtcattgtcagtacactatttttaattactt
ctcaacatcaagcacaagcagcagaaaatacaaatacttcagataaaatc
tcggaaaatcaaaataataatgcaactacaactcagccacctaaggatac
aaatcaaacacaacctgctacgcaaccagcaaacactgcgaaaaactatc
ctgcagcggatgaatcacttaaagatgcaattaaagatcctgcattagaa
aataaagaacatgatataggtccaagagaacaagtcaatttccagttatt
agataaaaacaatgaaacgcagtactatcactttttcagcatcaaagatc
cagcagatgtgtattacactaaaaagaaagcagaagttgaattagacatc
aatactgcttcaacatggaagaagtttgaagtctatgaaaacaatcaaaa
attgccagtgagacttgtatcatatagtcctgtaccagaagaccatgcct
atattcgattcccagtttcagatggcacacaagaattgaaaattgtttct
tcgactcaaattgatgatggagaagaaacaaattatgattatactaaatt
agtatttgctaaacctatttataacgatccttcacttgtaaaatcagata
caaatgatgcagtagtaacgaatgatcaatcaagttcagtcgcaagtaat
caaacaaacacgaatacatctaatcaaaatatatcaacgatcaacaatgc
taataatcaaccgcaggcaacgaccaatatgagtcaacctacacaaccaa
aatcgtcaacgaatgcagatcaagcgtcaagccaaccagctcatgaaaca
aattctaatggtaatactaacgataaaacgaatgagtcaagtaatcagtc
ggatgttaatcaacagtatccaccagcagatgaatcactacaagatgcaa
ttaaaaacccggctatcatcgataaagaacatacagctgataattggcga
ccaattgattttcaaatgaaaaatgataaaggtgaaagacagttctatca
ttatgctagtactgttgaaccagcaactgtcatttttacaaaaacaggac
caataattgaattaggtttaaagacagcttcaacatggaagaaatttgaa
gtttatgaaggtgacaaaaagttaccagtcgaattagtatcatatgattc
tgataaagattatgcctatattcgtttcccagtatctaatggtacgagag
aagttaaaattgtgtcatctattgaatatggtgagaacatccatgaagac
tatgattatacgctaatggtctttgcacagcctattactaataacccaga
cgactatgtggatgaagaaacatacaatttacaaaaattattagctccgt
atcacaaagctaaaacgttagaaagacaagtttatgaattagaaaaatta
caagagaaattgccagaaaaatataaggcggaatataaaaagaaattaga
tcaaactagagtagagttagctgatcaagttaaatcagcagtgacggaat
ttgaaaatgttacacctacaaatgatcaattaacagatttacaagaagcg
cattttgttgtttttgaaagtgaagaaaatagtgagtcagttatggacgg
ctttgttgaacatccattctatacagcaactttaaatggtcaaaaatatg
tagtgatgaaaacaaaggatgacagttactggaaagatttaattgtagaa
ggtaaacgtgtcactactgtttctaaagatcctaaaaataattctagaac
gctgattttcccatatatacctgacaaagcagtttacaatgcgattgtta
aagtcgttgtggcaaacattggttatgaaggtcaatatcatgtcagaatt
ataaatcaggatatcaatacaaaagatgatgatacatcacaaaataacac
gagtgaaccgctaaatgtacaaacaggacaagaaggtaaggttgctgata
cagatgtagctgaaaatagcagcactgcaacaaatcctaaagatgcgtct
gataaagcagatgtgatagaaccagagtctgacgtggttaaagatgctga
taataatattgataaagatgtgcaacatgatgttgatcatttatccgata
tgtcggataataatcacttcgataaatatgatttaaaagaaatggatact
caaattgccaaagatactgatagaaatgtggataaagatgccgataatag
cgttggtatgtcatctaatgtcgatactgataaagactctaataaaaata
aagacaaagtcatacagctgaatcatattgccgataaaaataatcatact
ggaaaagcagcaaagcttgacgtagtgaaacaaaattataataatacaga
caaagttactgacaaaaaaacaactgaacatctgccgagtgatattcata
aaactgtagataaaacagtgaaaacaaaagaaaaagccggcacaccatcg
aaagaaaacaaacttagtcaatctaaaatgctaccaaaaactggagaaac
aacttcaagccaatcatggtggggcttatatgcgttattaggtatgttag
ctttattcattcctaaattcagaaaagaatctaaataa

A number of homologous Staphylococcus IsdH amino acid and nucleotide sequences are available from Genbank. Similarly, a number of homologous IsdH amino acid and nucleotide sequences from other Staphylococcus species are also available from Genbank.

Any one or more of these IsdH sequences, whether now known or hereafter identified, can be used in accordance with the present invention. In certain embodiments, the homologous amino acid sequences comprise at least about 80 percent identity, at least about 85 percent identity, at least about 90 percent identity, or at least about 95 percent identity to the full length IsdH amino acid sequence provided above. In other embodiments, the homologous amino acid sequences comprise at least about 80 percent identity, at least about 85 percent identity, at least about 90 percent identity, or at least about 95 percent identity to residues 41-864 of the IsdH amino acid sequence provided above.

The use of IsdH polypeptide fragments is also contemplated, including fragments containing either linear or conformational epitopes. Such fragments may include at least 20, at least 30, at least 40, at least 50, at least 60, at least 70, at least 80, at least 90, or at least 100 contiguous amino acids.

Clumping Factor A (ClfA) is a cell surface-associated protein implicated in virulence. ClfA promotes bacterial attachment exclusively to the gamma-chain of human fibrinogen, and induces formation of bacterial clumps, which diminish the ability of group IIA phospholipase A2 to cause bacterial phospholipid hydrolysis and killing. ClfA significantly decreases macrophage phagocytosis possibly due to the clumps, clumped bacteria being too large to be phagocytosed. ClfA is a dominant factor responsible for human platelet aggregation, which may be an important mechanism for initiating infective endocarditis. It also enhances spleen cell proliferative response in vitro, contributing significantly to the immunostimulatory activity of S. aureus.

An exemplary ClfA has the amino acid sequence of the sequence shown below (SEQ ID NO: 10):

Staphylococcus aureus NCTC 8325 (Genbank Accession Q2G015, which is hereby incorporated by reference in its entirety)

| | |
|---|---|
| 1 | MNMKKKEKHA IRKKSIGVAS VLVGTLIGFG LLSSKEADAS ENSVTQSDSA SNESKSNDSS |
| 61 | SVSAAPKTDD TNVSDTKTSS NTNNGETSVA QNPAQQETTQ SSSTNATTEE TPVTGEATTT |
| 121 | TTNQANTPAT TQSSNTNAEE LVNQTSNETT SNDTNTVSSV NSPQNSTNAE NVSTTQDTST |
| 181 | EATPSNNESA PQSTDASNKD VVNQAVNTSA PRMRAFSLAA VAADAPVAGT DITNQLTNVT |
| 241 | VGIDSGTTVY PHQAGYVKLN YGFSVPNSAV KGDTFKITVP KELNLNGVTS TAKVPPIMAG |
| 301 | DQVLANGVID SDGNVIYTFT DYVNTKDDVK ATLTMPAYID PENVKKTGNV TLATGIGSTT |
| 361 | ANKTVLVDYE KYGKFYNLSI KGTIDQIDKT NNTYRQTIYV NPSGDNVIAP VLTGNLKPNT |
| 421 | DSNALIDQQN TSIKVYKVDN AADLSESYFV NPENFEDVTN SVNITFPNPN QYKVEFNTPD |
| 481 | DQITTPYIVV VNGHIDPNSK GDLALRSTLY GYNSNIIWRS MSWDNEVAFN NGSGSGDGID |
| 541 | KPVVPEQPDE PGEIEPIPED SDSDPGSDSG SDSNSDSGSD SGSDSTSDSG SDSASDSDSA |
| 601 | SDSDSASDSD SASDSDSASD SDSDNDSDSD SDSDSDSDSD SDSDSDSDSD SDSDSDSDSD |
| 661 | SDSDSDSDSD SDSDSDSDSD SDSDSDSDSD SDSDSDSDSD SDSDSDSDSD SDSDSDSDSD |
| 721 | SDSDSDSDSD SDSDSDSDSD SDSDSDSDSD SDSDSDSDSD SDSDSDSASD SDSDSDSDSD |
| 781 | SDSDSDSDSD SDSDSDSDSD SDSDSDSESD SDSDSDSDSD SDSDSDSDSD SASDSDSGSD |
| 841 | SDSSSDSDSE SDSNSDSESV SNNNVVPPNS PKNGTNASNK NEAKDSKEPL PDTGSEDEAN |
| 901 | TSLIWGLLAS IGSLLLFRRK KENKDKK |

Amino acids 1-39 (italics) represent a likely signal peptide, amino acids 890-894 (underline) represents a cell wall anchor domain having an LPXTG-motif, and the region from 895-923 represents a propeptide sequence that is enzymatically cleaved, e.g., by a sortase. The ligand binding A region constitutes amino acids 40-542. The nucleotide sequence encoding the above-identified ClfA is provided at Genbank Accession NC_007795, which is hereby incorporated by reference in its entirety, and set forth below (SEQ ID NO: 11).

atgaatatgaagaaaaaagaaaaacacgcaattcggaaaaaatcgattgg
cgtggcttcagtgcttgtaggtacgttaatcggttttggactactcagca
gtaaagaagcagatgcaagtgaaaatagtgttacgcaatctgatagcgca
agtaacgaaagcaaaagtaatgattcaagtagcgttagtgctgcacctaa
aacagacgacacaaacgtgagtgatactaaaacatcgtcaaacactaata
atggcgaaacgagtgtggcgcaaaatccagcacaacaggaaacgacacaa
tcatcatcaacaaatgcaactacggaagaaacgccggtaactggtgaagc
tactactacgacaacgaatcaagctaatacaccggcaacaactcaatcaa
gcaatacaaatgcggaggaattagtgaatcaaacaagtaatgaaacgact
tctaatgatactaatacagtatcatctgtaaattcacctcaaaattctac
aaatgcggaaaatgtttcaacaacgcaagatacttcaactgaagcaacac
cttcaaacaatgaatcagctccacagagtacagatgcaagtaataaagat
gtagttaatcaagcggttaatacaagtgcgcctagaatgagagcatttag
tttagcggcagtagctgcagatgcaccggtagctggcacagatattacga
atcagttgacgaatgtgacagttggtattgactctggtacgactgtgtat
ccgcaccaagcaggttatgtcaaactgaattatggtttttcagtgcctaa
ttctgctgttaaaggtgacacattcaaaataactgtacctaaagaattaa
acttaaatggtgtaacttcaactgctaaagtgccaccaattatggctgga
gatcaagtattggcaaatggtgtaatcgatagtgatggtaatgttattta
tacatttacagactatgtaaatactaaagatgatgtaaaagcaactttga
ccatgcccgcttatattgaccctgaaaatgttaaaaagacaggtaatgtg
acattggctactggcataggtagtacaacagcaaacaaaacagtattagt
agattatgaaaaatatggtaagttttataacttatctattaaaggtacaa
ttgaccaaatcgataaaacaaataatacgtatcgtcagacaatttatgtc
aatccaagtggagataacgttattgcgccggttttaacaggtaatttaaa
accaaatacggatagtaatgcattaatagatcagcaaaatacaagtatta
aagtatataaagtagataatgcagctgatttatctgaaagttactttgtg
aatccagaaaactttgaggatgtcactaatagtgtgaatattacattccc
aaatccaaatcaatataaagtagagtttaatacgcctgatgatcaaatta
caacaccgtatatagtagttgttaatggtcatattgatccgaatagcaaa
ggtgatttagctttacgttcaactttatatgggtataactcgaatataat
ttggcgctctatgtcatgggacaacgaagtagcatttaataacggatcag
gttctggtgacggtatcgataaaccagttgttcctgaacaacctgatgag
cctggtgaaattgaaccaattccagaggattcagattctgacccaggttc
agattctggcagcgattctaattcagatagcggttcagattcgggtagtg
attctacatcagatagtggttcagattcagcgagtgattcagattcagca
agtgattcagactcagcgagtgattcagattcagcaagcgattccgactc
agcgagcgattccgactcagacaatgactcggattcagatagcgattctg
actcagacagtgactcagattccgacagtgactcagattcagatagcgat
tctgactcagacagtgactcggattcagatagcgattcagattcagatag
cgattcagattccgacagtgattccgactcagacagcgattctgactccg
acagtgattccgactcagacagcgattcagattccgacagtgattccgac
tcagatagcgattccgactcagatagcgactcagattcagacagcgattc
agattcagacagcgattcagattcagatagcgattcagattccgacagtg
actcagattccgacagtgactcggattcagatagcgattcagattccgac
agtgactcagattccgacagtgactcagactcagacagtgattcggattc
agcgagtgattcggattcagatagtgattccgactccgacagtgactcgg
attcagatagcgactcagactcggatagcgactcggattcagatagcgat
tcggactcagatagcgattcagaatcagacagcgattcagattcagacag
cgactcagacagtgactcagattcagatagtgactcggattcagcgagtg
attcagactcaggtagtgactccgattcatcaagtgattccgactcagaa
agtgattcaaatagcgattccgagtcagtttctaacaataatgtagttcc
gcctaattcacctaaaaatggtactaatgcttctaataaaaatgaggcta
aagatagtaaagaaccattaccagatacaggttctgaagatgaagcaaat
acgtcactaatttggggattattagcatcaataggttcattactactttt
cagaagaaaaaaagaaaataaagataagaaataa

A number of homologous Staphylococcus ClfA amino acid and nucleotide sequences are available from Genbank. Similarly, a number of homologous ClfA amino acid and nucleotide sequences from other Staphylococcus species are also available from Genbank.

Any one or more of these ClfA sequences, whether now known or hereafter identified, can be used in accordance with the present invention. In certain embodiments, the homologous amino acid sequences comprise at least about 80 percent identity, at least about 85 percent identity, at least about 90 percent identity, or at least about 95 percent identity to the full length ClfA amino acid sequence provided above. In other embodiments, the homologous amino acid sequences comprise at least about 80 percent identity, at least about 85 percent identity, at least about 90 percent identity, or at least about 95 percent identity to residues 40-542 of the ClfA amino acid sequence provided above.

The use of ClfA polypeptide fragments is also contemplated, including fragments containing either linear or conformational epitopes. Such fragments may include at least 20, at least 30, at least 40, at least 50, at least 60, at least 70, at least 80, at least 90, or at least 100 contiguous amino acids.

Clumping Factor B (ClfB) is a cell surface-associated protein implicated in virulence by promoting bacterial attachment to both alpha- and beta-chains of human fibrinogen and inducing the formation of bacterial clumps.

An exemplary ClfB has the amino acid sequence of the sequence shown below (SEQ ID NO: 12):

Staphylococcus aureus NCTC 8325 (Genbank Accession Q2FUY2, which is hereby incorporated by reference in its entirety)

| | |
|---|---|
| 1 | MKKRIDYLSN KQNKYSIRRF TVGTTSVIVG ATILFGIGNH QAQASEQSND TTQSSKNNAS |
| 61 | ADSEKNNMIE TPQLNTTAND TSDISANTNS ANVDSTTKPM STQTSNTTTT EPASTNETPQ |
| 121 | PTAIKNQATA AKMQDQTVPQ EANSQVDNKT TNDANSIATN SELKNSQTLD LPQSSPQTIS |
| 181 | NAQGTSKPSV RTRAVRSLAV AEPVVNAADA KGTNVNDKVT ASNFKLEKTT FDPNQSGNTF |
| 241 | MAANFTVTDK VKSGDYFTAK LPDSLTGNGD VDYSNSNNTM PIADIKSTNG DVVAKATYDI |
| 301 | LTKTYTFVFT DYVNNKENIN GQFSLPLFTD RAKAPKSGTY DANINIADEM FNNKITYNYS |
| 361 | SPIAGIDKPN GANISSQIIG VDTASGQNTY KQTVFVNPKQ RVLGNTWVYI KGYQDKIEES |
| 421 | SGKVSATDTK LRIFEVNDTS KLSDSYYADP NDSNLKEVTD QFKNRIYYEH PNVASIKFGD |
| 481 | ITKTYVVLVE GHYDNTGKNL KTQVIQENVD PVTNRDYSIF GWNNENVVRY GGGSADGDSA |
| 541 | VNPKDPTPGP PVDPEPSPDP EPEPTPDPEP SPDPEPEPSP DPDPDSDSDS DSGSDSDSGS |
| 601 | DSDSESDSDS DSDSDSDSDS DSESDSDSES DSESDSDSDS DSDSDSDSDS DSDSDSDSDS |
| 661 | DSDSDSDSDS DSDSDSDSDS DSDSDSDSDS DSDSDSDSDS DSDSDSDSDS DSDSDSDSDS |
| 721 | DSDSDSDSDS DSDSDSDSDS DSDSDSDSDS DSDSDSDSDS DSDSDSDSDS DSDSDSDSDS |
| 781 | DSDSDSDSDS DSDSDSDSDS DSDSRVTPPN NEQKAPSNPK GEVNHSNKVS KQHKTDALPE |
| 841 | TGDKSENTNA TLFGAMMALL GSLLLFRKRK QDHKEKA |

Amino acids 1-44 (italics) represent a likely signal peptide, amino acids 838-842 (underline) represents a cell wall anchor domain having an LPXTG-motif, and the region from 843-877 represents a propeptide sequence that is enzymatically cleaved, e.g., by a sortase. The ligand binding A region constitutes amino acids 45-542. The nucleotide sequence encoding the above-identified ClfB is provided at Genbank Accession NC_007795, which is hereby incorporated by reference in its entirety, and set forth below (SEQ ID NO: 13).

ttgaaaaaaagaattgattatttgtcgaataagcagaataagtattcgat
tagacgttttacagtaggtaccacatcagtaatagtaggggcaactatac
tatttgggataggcaatcatcaagcacaagcttcagaacaatcgaacgat
acaacgcaatcttcgaaaaataatgcaagtgcagattccgaaaaaaacaa
tatgatagaaacacctcaattaaatacaacggctaatgatacatctgata
ttagtgcaaacacaaacagtgcgaatgtagatagcacaacaaaaccaatg
tctacacaaacgagcaataccactacaacagagccagcttcaacaaatga
aacacctcaaccgacggcaattaaaaatcaagcaactgctgcaaaaatgc
aagatcaaactgttcctcaagaagcaaattctcaagtagataataaaaca
acgaatgatgctaatagcatagcaacaaacagtgagcttaaaaattctca
aacattagatttaccacaatcatcaccacaaacgatttccaatgcgcaag
gaactagtaaaccaagtgttagaacgagagctgtacgtagtttagctgtt
gctgaaccggtagtaaatgctgctgatgctaaaggtacaaatgtaaatga
taaagttacggcaagtaatttcaagttagaaaagactacatttgacccta
atcaaagtggtaacacatttatggcggcaaattttacagtgacagataaa
gtgaaatcaggggattattttacagcgaagttaccagatagtttaactgg
taatggagacgtggattattctaattcaaataatacgatgccaattgcag
acattaaaagtacgaatggcgatgttgtagctaaagcaacatatgatatc
ttgactaagacgtatacatttgtctttacagattatgtaaataataaaga
aaatattaacggacaattttcattacctttatttacagaccgagcaaagg
cacctaaatcaggaacatatgatgcgaatattaatattgcggatgaaatg
tttaataataaaattacttataactatagttcgccaattgcaggaattga
taaaccaaatggcgcgaacatttcttctcaaattattggtgtagatacag
cttcaggtcaaaacacatacaagcaaacagtatttgttaaccctaagcaa
cgagttttaggtaatacgtgggtgtatattaaaggctaccaagataaaat
cgaagaaagtagcggtaaagtaagtgctacagatacaaaactgagaattt
ttgaagtgaatgatacatctaaattatcagatagctactatgcagatcca
aatgactctaaccttaaagaagtaacagaccaatttaaaaatagaatcta
ttatgagcatccaaatgtagctagtattaaatttggtgatattactaaaa
catatgtagtattagtagaagggcattacgacaatacaggtaagaactta
aaaactcaggttattcaagaaaatgttgatcctgtaacaaatagagacta
cagtattttcggttggaataatgagaatgttgtacgttatggtggtggaa
gtgctgatggtgattcagcagtaaatccgaaagacccaactccagggccg
ccggttgacccagaaccaagtccagacccagaaccagaaccaacgccaga
tccagaaccaagtccagacccagaaccggaaccaagcccagacccggatc
cggattcggattcagacagtgactcaggctcagacagcgactcaggttca
gatagcgactcagaatcagatagcgattcggattcagacagtgattcaga
ttcagacagcgactcagaatcagatagcgactcagaatcagatagtgagt
cagattcagacagtgactcggactcagacagtgattcagactcagatagc
gattcagactcagatagcgattcagactcagacagcgattcagattcaga
cagcgactcagattcagacagcgactcagactcagatagcgactcagact
cagacagcgactcagattcagatagcgattcagactcagacagcgactca
gactcagacagcgactcagactcagatagcgactcagattcagatagcga
ttcagactcagacagcgactcagattcagatagcgattcggactcagaca
gcgattcagattcagacagcgactcagactcggatagcgattcagattca
gatagcgattcggattcagacagtgattcagattcagacagcgactcaga
ctcggatagcgactcagactcagacagcgattcagactcagatagcgact
cagactcggatagcgactcggattcagatagcgactcagactcagatagt
gactccgattcaagagttacaccaccaaataatgaacagaaagcaccatc
aaatcctaaaggtgaagtaaaccattctaataaggtatcaaaacaacaca
aaactgatgctttaccagaaacaggagataagagcgaaaacacaaatgca
actttatttggtgcaatgatggcattattaggatcattactattgtttag
aaaacgcaagcaagatcataaagaaaaagcgtaa

A number of homologous Staphylococcus ClfB amino acid and nucleotide sequences are available from Genbank. Similarly, a number of homologous ClfB amino acid and nucleotide sequences from other Staphylococcus species are also available from Genbank.

Any one or more of these ClfB sequences, whether now known or hereafter identified, can be used in accordance with the present invention. In certain embodiments, the homologous amino acid sequences comprise at least about 80 percent identity, at least about 85 percent identity, at least about 90 percent identity, or at least about 95 percent identity to the full length ClfB amino acid sequence provided above. In other embodiments, the homologous amino acid sequences comprise at least about 80 percent identity, at least about 85 percent identity, at least about 90 percent identity, or at least about 95 percent identity to residues 45-542 of the ClfB amino acid sequence provided above.

The use of ClfB polypeptide fragments is also contemplated, including fragments containing either linear or conformational epitopes. Such fragments may include at least 20, at least 30, at least 40, at least 50, at least 60, at least 70, at least 80, at least 90, or at least 100 contiguous amino acids.

Fibronectin Binding Protein A (FnbpA) possesses multiple, substituting fibronectin (Fn) binding regions, each capable of conferring adherence to both soluble and immobilized forms of Fn. This confers to S. aureus the ability to invade endothelial cells both in vivo and in vitro, without requiring additional factors, although in a slow and inefficient way through actin rearrangements in host cells. This invasion process is mediated by integrin α5/β1. FnbpA promotes bacterial attachment to both soluble and immobilized forms of fibrinogen (Fg) by means of a unique binding site localized within the 17 C-terminal residues of the gamma-chain of human Fg. Both plasma proteins (Fn and Fg) function as a bridge between bacterium and host cell. FnbpA promotes attachment to immobilized elastin peptides in a dose-dependent and saturable manner, and both full-length and segments of immobilized human tropoelastin at multiple sites in a dose and pH-dependent manner. FnbpA also promotes attachment to and aggregation of activated platelets independently of other S. aureus surface molecules. FnbpA is a critical mediator implicated in the induction of experimental endocarditis in rats with catheter-induced aortic vegetations, promoting both colonization and persistence of the bacterium into the host.

An exemplary FnbpA has the amino acid sequence of the sequence shown below (SEQ ID NO: 14):

Staphylococcus aureus NCTC 8325 (Genbank Accession P14738, which is hereby incorporated by reference in its entirety)

| | |
|---|---|
| 1 | MKNNLRYGIR KHKLGAASVF LGTMIVVGMG QDKEAAASEQ KTTTVEENGN SATDNKTSET |
| 61 | QTTATNVNHI EETQSYNATV TEQPSNATQV TTEEAPKAVQ APQTAQPANI ETVKEEVVKE |
| 121 | EAKPQVKETT QSQDNSGDQR QVDLTPKKAT QNQVAETQVE VAQPRTASES KPRVTRSADV |
| 181 | AEAKEASNAK VETGTDVTSK VTVEIGSIEG HNNTNKVEPH AGQRAVLKYK LKFENGLHQG |
| 241 | DYFDFTLSNN VNTHGVSTAR KVPEIKNGSV VMATGEVLEG GKIRYTFTND IEDKVDVTAE |
| 301 | LEINLFIDPK TVQTNGNQTI TSTLNEEQTS KELDVKYKDG IGNYYANLNG SIETFNKANN |
| 361 | RFSHVAFIKP NNGKTTSVTV TGTLMKGSNQ NGNQPKVRIF EYLGNNEDIA KSVYANTTDT |
| 421 | SKFKEVTSNM SGNLNLQNNG SYSLNIENLD KTYVVHYDGE YLNGTDEVDF RTQMVGHPEQ |
| 481 | LYKYYYDRGY TLTWDNGLVL YSNKANGNEK NGPIIQNNKF EYKEDTIKET LTGQYDKNLV |
| 541 | TTVEEEYDSS TLDIDYHTAI DGGGGYVDGY IETIEETDSS AIDIDYHTAV DSEAGHVGGY |
| 601 | TESSEESNPI DFEESTHENS KHHADVVEYE EDTNPGGGQV TTESNLVEFD EESTKGIVTG |
| 661 | AVSDHTTVED TKEYTTESNL IELVDELPEE HGQAQGPVEE ITKNNHHISH SGLGTENGHG |
| 721 | NYDVIEEIEE NSHVDIKSEL GYEGGQNSGN QSFEEDTEED KPKYEQGGNI VDIDFDSVPQ |
| 781 | IHGQNKGNQS FEEDTEKDKP KYEHGGNIID IDFDSVPHIH GFNKHTEIIE EDTNKDKPSY |
| 841 | QFGGHNSVDF EEDTLPKVSG QNEGQQTIEE DTTPPIVPPT PPTPEVPSEP ETPTPPTPEV |
| 901 | PSEPETPTPP TPEVPSEPET PTPPTPEVPA EPGKPVPPAK EEPKKPSKPV EQGKVVTPVI |
| 961 | EINEKVKAVA PTKKPQSKKS ELPETGGEES TNKGMLFGGL FSILGLALLR RNKKNHKA |

Amino acids 1-36 (italics) represent a likely signal peptide, amino acids 982-986 (underline) represents a cell wall anchor domain having an LPXTG-motif, and the region from 987-1018 represents a propeptide sequence that is enzymatically cleaved, e.g., by a sortase. The ligand binding A region constitutes amino acids 37-511; this region also includes sequence similarity to fibrinogen/elastin/tropoelastin-binding domains. The nucleotide sequence encoding the above-identified FnbpA is provided at Genbank Accession NC_007795, which is hereby incorporated by reference in its entirety, and set forth below (SEQ ID NO: 15).

atgggacaagacaaagaagctgcagcatcagaacaaaagacaactacagt
agaagaaaatgggaattcagctactgataataaaacaagtgaaacacaaa
caactgcaactaacgttaatcatatagaagaaactcaatcatataacgca
acagtaacagaacaaccgtcaaacgcaacacaagtaacaactgaagaagc
accaaaagcagtacaagcaccacaaactgcacaaccagcaaatatagaaa
cagttaaagaagaggtagttaaggaagaagcgaaacctcaagttaaggaa
acaacacaatctcaagacaatagcggagatcaaagacaagtagatttaac
acctaaaaaggctacacaaaatcaagtcgcagaaacacaagttgaagtgg
cacagccaagaacggcatcagaaagtaagccacgtgtgacaagatcagca
gatgtagcggaagctaaggaagctagtaacgcgaaagtggaaacgggtac
agatgtaacaagtaaagttacagtagaaattggttctattgaggggcata
acaatacaaataaagtagaacctcatgcaggacaacgagcggtactaaaa
tataagttgaaatttgagaatggtttacatcaaggtgactactttgactt
tactttatcaaataatgtaaatacgcatggcgtatcaactgctagaaaag
taccagaaattaaaaatggttcagtcgtaatggcgacaggtgaagtttta
gaaggtggaaagattagatatacatttacaaatgatattgaagataaggt
tgatgtaacggctgaactagaaattaatttatttattgatcctaaaactg
tacaaactaatggaaatcaaactataacttcaacactaaatgaagaacaa
acttcaaaggaattagatgttaaatataaagatggtattgggaattatta
tgccaatttaaatggatcgattgagacatttaataaagcgaataatagat
tttcgcatgttgcatttattaaacctaataatggtaaaacgacaagtgtg
actgttactggaactttaatgaaaggtagtaatcagaatggaaatcaacc
aaaagttaggatatttgaatacttgggtaataatgaagacatagcgaaga
gtgtatatgcaaatacgacagatacttctaaatttaaagaagtcacaagt
aatatgagtgggaatttgaatttacaaaataatggaagctattcattgaa
tatagaaaatctagataaaacttatgttgttcactatgatggagagtatt
taaatggtactgatgaagttgattttagaacacaaatggtaggacatcca
gagcaactttataagtattattatgatagaggatataccttaacttggga
taatggtttagttttatacagtaataaagcgaacggaaatgagaaaaatg
gtccgattattcaaaataataaatttgaatataaagaagatacaattaaa
gaaactcttacaggtcaatatgataagaatttagtaactactgttgaaga
ggaatatgattcatcaactcttgacattgattaccacacagctatagatg
gtggaggtggatatgttgatggatacattgaaacaatagaagaaacggat
tcatcagctattgatatcgattaccatactgctgtggatagcgaagcagg
tcacgttggaggatacactgagtcctctgaggaatcaaatccaattgact
ttgaagaatctacacatgaaaattcaaaacatcacgctgatgttgttgaa
tatgaagaagatacaaacccaggtggtggtcaggttactactgagtctaa
cttagttgaatttgacgaagagtctacaaaaggtattgtaactggcgcag
tgagcgatcatacaacagttgaagatacgaaagaatatacaactgaaagt
aatctgattgaattagtggatgaattacctgaagagcatggtcaagcaca
aggaccagtcgaggaaattactaaaaacaatcatcatatttctcattctg
gtttaggaactgaaaatggtcacgggaattatgacgtgattgaagaaatc
gaagaaaatagccacgttgatattaagagtgaattaggttatgaaggtgg
ccaaaatagcggtaaccagtcattcgaggaagacacagaagaagacaaac
ctaaatatgaacaaggtggcaatatcgtagatatcgattttgatagtgta
cctcaaattcatggtcaaaataaaggtaatcagtcattcgaggaagatac
agaaaaagacaaacctaagtatgaacatggcggtaacatcattgatatcg
acttcgacagtgtgccacatattcacggattcaataagcacactgaaatt
attgaagaagatacaaataaagataaaccaagttatcaattcggtggaca
caatagtgttgactttgaagaagatacacttccaaaagtaagcggccaaa
atgaaggtcaacaaacgattgaagaagatacaacacctccaatcgtgcca
ccaacgccaccgacaccagaagtaccaagtgagccggaaacaccaacgcc
accaacaccagaagtaccaagtgagccggaaacaccaacaccaccgacac
cagaagtgccgagtgagccagaaactccaacaccgccaacaccagaggta
ccagctgaacctggtaaaccagtaccacctgccaaagaagaacctaaaaa
gccttctaaaccagtggaacaaggtaaagtagtaacacctgttattgaaa
tcaatgaaaaggttaaagcagtggcaccaactaaaaaaccacaatctaag
aaatctgaactacctgaaacaggtggagaagaatcaacaaacaaaggtat
gttgttcggcggattattcagcattctaggtttagcattattacgcagaa
ataaaaagaatcacaaagcataa

A number of homologous Staphylococcus FnbpA amino acid and nucleotide sequences are available from Genbank. Similarly, a number of homologous FnbpA amino acid and nucleotide sequences from other Staphylococcus species are also available from Genbank.

Any one or more of these FnbpA sequences, whether now known or hereafter identified, can be used in accordance with the present invention. In certain embodiments, the homologous amino acid sequences comprise at least about 80 percent identity, at least about 85 percent identity, at least about 90 percent identity, or at least about 95 percent identity to the full length FnbpA amino acid sequence provided above. In other embodiments, the homologous amino acid sequences comprise at least about 80 percent identity, at least about 85 percent identity, at least about 90 percent identity, or at least about 95 percent identity to residues 37-511 of the FnbpA amino acid sequence provided above.

The use of FnbpA polypeptide fragments is also contemplated, including fragments containing either linear or conformational epitopes. Such fragments may include at least 20, at least 30, at least 40, at least 50, at least 60, at least 70, at least 80, at least 90, or at least 100 contiguous amino acids.

Staphylococcus Complement Inhibitor (SCIN) is involved in countering the first line of host defense mechanisms. SCIN efficiently inhibits opsonization, phagocytosis and killing of Staphylococcus by human neutrophils. SCIN acts by binding and stabilizing human C3 convertases (C4b2a and C3bBb), leading to their inactivation, in which case the convertases are no longer able to cleave complement C3 and therefore prevent further C3b deposition on the bacterial surface and phagocytosis of the bacterium. SCIN also prevents C5a-induced neutrophil responses.

An exemplary SCIN has the amino acid sequence of the sequence shown below (SEQ ID NO: 16):

Staphylococcus aureus NCTC 8325 (Genbank Accession Q2FWV6, which is hereby incorporated by reference in its entirety)

| | |
|---|---|
| 1 | MKIRKSILAG TLAIVLASPL VTNLDKNEAQ ASTSLPTSNE YQNEKLANEL KSLLDELNVN |
| 61 | ELATGSLNTY YKRTIKISGL KAMYALKSKD FKKMSEAKYQ LQKIYNEIDE ALKSKY |

Amino acids 1-31 (italics) represent a likely signal peptide, and the region from 32-116 represents the secreted protein. The nucleotide sequence encoding the above-identified SCIN is provided at Genbank Accession NC_007795, which is hereby incorporated by reference in its entirety, and set forth below (SEQ ID NO: 17).

atgaaaattagaaaatctatacttgcgggaactttagcaatcgttttagc
atcaccactagtaactaatctagataaaaatgaggcacaagctagcacaa
gcttgccaacatcgaatgaatatcaaaacgaaaagttagctaatgaatta
aaatcgttattagatgaactaaatgttaatgaattagctactggaagttt
aaacacttattataagcgaactataaaaatttcaggtctaaaagcaatgt
atgctcttaagtcaaaagactttaagaaaatgtcagaagcaaaatatcaa
cttcaaaagatttataacgaaattgacgaagcactaaaaagtaaatatta
a

A number of homologous Staphylococcus SCIN amino acid and nucleotide sequences are available from Genbank. Similarly, a number of homologous SCIN amino acid and nucleotide sequences from other Staphylococcus species are also available from Genbank.

Any one or more of these SCIN sequences, whether now known or hereafter identified, can be used in accordance with the present invention. In certain embodiments, the homologous amino acid sequences comprise at least about 80 percent identity, at least about 85 percent identity, at least about 90 percent identity, or at least about 95 percent identity to the full length SCIN amino acid sequence provided above. In other embodiments, the homologous amino acid sequences comprise at least about 80 percent identity, at least about 85 percent identity, at least about 90 percent identity, or at least about 95 percent identity to residues 32-116 of the SCIN amino acid sequence provided above.

The use of SCIN polypeptide fragments is also contemplated, including fragments containing either linear or conformational epitopes. Such fragments may include at least 20, at least 30, at least 40, at least 50, at least 60, at least 70, or at least 80 contiguous amino acids.

Chemotaxis Inhibitory Protein of Staphylococcus (CHIPS) is involved in countering the first line of host defense mechanisms. Specifically, CHIPS inhibits the response of human neutrophils and monocytes to complement anaphylatoxin C5a and formylated peptides, like N-formyl-methionyl-leucyl-phenylalanine (fMLF). CHIPS acts by binding directly to the C5a receptor (C5aR) and formylated peptide receptor (FPR), thereby blocking the C5a- and fMLF-induced calcium responses. CHIPS also prevents phagocytosis of the bacterium.

An exemplary CHIPS has the amino acid sequence of the sequence shown below (SEQ ID NO: 18):

Staphylococcus aureus NCTC 8325 (Genbank Accession Q2FWV5, which is hereby incorporated by reference in its entirety)

| | |
|---|---|
| 1 | MKKKLATTVL ALSFLTAGIS THHHSAKAFT FEPFPTNEEI ESNKKLLEKE KAYKESFKNS |
| 61 | GLPTTLGKLD ERLRNYLKKG TKNSAQFEKM VILTENKGYY TVYLNTPLAE DRKNVELLGK |
| 121 | MYKTYFFKKG ESKSSYVING PGKTNEYAY |

Amino acids 1-28 (italics) represent a likely signal peptide, and the region from 29-149 represents the mature protein. The region from 29-34 possesses FPR blocking activity, and the region from 59-149 possesses C5aR blocking activity. The nucleotide sequence encoding the above-identified CHIPS is provided at Genbank Accession NC_007795, which is hereby incorporated by reference in its entirety, and set forth below (SEQ ID NO: 19).

atgaaaaagaaattagcaacaacagttttagcattaagttttttaacggc
aggaatcagtacacaccatcattcagcgaaagcttttacttttgaaccgt
ttcctacaaatgaagaaatagaatcaaataagaaattgttagagaaagaa
aaagcttataaagaatcatttaaaaatagtggtcttcctacaacactagg
aaaattagatgaacgtttgagaaattatttaaagaaaggcacaaaaaatt
ctgctcaatttgaaaaaatggttattttaactgaaaataaaggttactat
acagtatatctgaatacaccacttgctgaagatagaaaaaatgttgagtt
actaggtaaaatgtataaaacatacttctttaaaaaaggagagtctaaat
catcttatgtaattaatggtcctggaaaaactaatgaatatgcatactaa

A number of homologous Staphylococcus CHIPS amino acid and nucleotide sequences are available from Genbank. Similarly, a number of homologous CHIPS amino acid and nucleotide sequences from other Staphylococcus species are also available from Genbank.

Any one or more of these CHIPS sequences, whether now known or hereafter identified, can be used in accordance with the present invention. In certain embodiments, the homologous amino acid sequences comprise at least about 80 percent identity, at least about 85 percent identity, at least about 90 percent identity, or at least about 95 percent identity to the full length CHIPS amino acid sequence provided above. In other embodiments, the homologous amino acid sequences comprise at least about 80 percent identity, at least about 85 percent identity, at least about 90 percent identity, or at least about 95 percent identity to residues 29-149 of the CHIPS amino acid sequence provided above.

The use of CHIPS polypeptide fragments is also contemplated, including fragments containing either linear or conformational epitopes. Such fragments may include at least 20, at least 30, at least 40, at least 50, at least 60, at least 70, at least 80, at least 90, or at least 100 contiguous amino acids.

α-Hemolysin (Hla) is secreted as a monomer, and thereafter self-assembles to form first a non-lytic oligomeric intermediate and then, a mushroom-shaped homoheptamer structure of 100 Angstroms in length and up to 100 Angstroms in diameter. After oligomerization and pore formation, the complex is translocated across the bilayer, probably via the Gly-rich domain of each strand. Hla oligomer binds to the membrane of eukaryotic cells resulting in the release of low-molecular weight molecules and leading to an eventual osmotic lysis. Heptamer oligomerization and pore formation is required for lytic activity.

An exemplary Hla has the amino acid sequence of the sequence shown below (SEQ ID NO: 20):

Staphylococcus aureus NCTC 8325 (Genbank Accession Q2G1X0, which is hereby incorporated by reference in its entirety)

| | |
|---|---|
| 1 | MKTRIVSSVT TTLLLGSILM NPVANAADSD INIKTGTTDI GSNTTVKTGD LVTYDKENGM |
| 61 | HKKVFYSFID DKNHNKKLLV IRTKGTIAGQ YRVYSEEGAN KSGLAWPSAF KVQLQLPDNE |
| 121 | VAQISDYYPR NSIDTKEYMS TLTYGFNGNV TGDDTGKIGG LIGANVSIGH TLKYVQPDFK |
| 181 | TILESPTDKK VGWKVIFNNM VNQNWGPYDR DSWNPVYGNQ LFMKTRNGSM KAADNFLDPN |
| 241 | KASSLLSSGF SPDFATVITM DRKASKQQTN IDVIYERVRD DYQLHWTSTN WKGTNTKDKW |
| 301 | IDRSSERYKI DWEKEEMTN |

Amino acids 1-26 (italics) represent a likely signal peptide, and the region from 27-319 represents the functional monomer. The nucleotide sequence encoding the above-identified Hla is provided at Genbank Accession NC_007795, which is hereby incorporated by reference in its entirety, and set forth below (SEQ ID NO: 21).

atgaaaacacgtatagtcagctcagtaacaacaacactattgctaggttc
catattaatgaatcctgtcgctaatgccgcagattctgatattaatatta
aaaccggtactacagatattggaagcaatactacagtaaaaacaggtgat
ttagtcacttatgataaagaaaatggcatgcacaaaaaagtattttatag
ttttatcgatgataaaaatcataataaaaaactgctagttattagaacga
aaggtaccattgctggtcaatatagagtttatagcgaagaaggtgctaac
aaaagtggtttagcctggccttcagcctttaaggtacagttgcaactacc
tgataatgaagtagctcaaatatctgattactatccaagaaattcgattg
atacaaaagagtatatgagtactttaacttatggattcaacggtaatgtt
actggtgatgatacaggaaaaattggcggccttattggtgcaaatgtttc
gattggtcatacactgaaatatgttcaacctgatttcaaaacaattttag
agagcccaactgataaaaaagtaggctggaaagtgatatttaacaatatg
gtgaatcaaaattggggaccatatgatagagattcttggaacccggtata
tggcaatcaacttttcatgaaaactagaaatggctctatgaaagcagcag
ataacttccttgatcctaacaaagcaagttctctattatcttcagggttt
tcaccagacttcgctacagttattactatggatagaaaagcatccaaaca
acaaacaaatatagatgtaatatacgaacgagttcgtgatgactaccaat
tgcactggacttcaacaaattggaaaggtaccaatactaaagataaatgg
atagatcgttcttcagaaagatataaaatcgattgggaaaaagaagaaat
gacaaattaa

A number of homologous Staphylococcus Hla amino acid and nucleotide sequences are available from Genbank. Similarly, a number of homologous Hla amino acid and nucleotide sequences from other Staphylococcus species are also available from Genbank.

Any one or more of these Hla sequences, whether now known or hereafter identified, can be used in accordance with the present invention. In certain embodiments, the homologous amino acid sequences comprise at least about 80 percent identity, at least about 85 percent identity, at least about 90 percent identity, or at least about 95 percent identity to the full length Hla amino acid sequence provided above. In other embodiments, the homologous amino acid sequences comprise at least about 80 percent identity, at least about 85 percent identity, at least about 90 percent identity, or at least about 95 percent identity to residues 27-319 of the Hla amino acid sequence provided above.

The use of Hla polypeptide fragments is also contemplated, including fragments containing either linear or conformational epitopes. Such fragments may include at least 20, at least 30, at least 40, at least 50, at least 60, at least 70, at least 80, at least 90, or at least 100 contiguous amino acids. In certain embodiments, the polypeptide fragments may retain their ability to form heptamers.

Extracellular Fibrinogen-binding Protein (Efb) binds to fibrinogen and inhibits the complement cascade by binding to the important protein, complement C3b. In particular, Efb inhibits the interaction of C3d with complement receptor 2 (CR2), which plays an important role in B cell activation and maturation. The C-terminal domain of Efb efficiently blocks this C3d-CR2 interaction, and prevents the CR2-mediated stimulation of B cells. Both the N-terminal half and the C-terminal half of Efb contain fibrinogen binding domains.

An exemplary Efb has the amino acid sequence of the sequence shown below (SEQ ID NO: 22):

Staphylococcus aureus NCTC 8325 (Genbank Accession Q2G1X0, which is hereby incorporated by reference in its entirety)

| | |
|---|---|
| 1 | MKNKLIAKSL LTLAAIGITT TTIASTADAS EGYGPREKKP VSINHNIVEY NDGTFKYQSR |
| 61 | PKFNSTPKYI KFKHDYNILE FNDGTFEYGA RPQFNKPAAK TDATIKKEQK LIQAQNLVRE |
| 121 | FEKTHTVSAH RKAQKAVNLV SFEYKVKKMV LQERIDNVLK QGLVK |

The nucleotide sequence encoding the above-identified Efb is provided at Genbank Accession NC_007795, which is hereby incorporated by reference in its entirety, and set forth below (SEQ ID NO: 23).

atgaaaaataaattgatagcaaaatctttattaacattagcggcaatagg
tattactacaactacaattgcgtcaacagcagatgcgagcgaaggatacg
gtccaagagaaaagaaaccagtgagtattaatcacaatatcgtagagtac
aatgatggtacttttaaatatcaatctagaccaaaatttaactcaacacc
taaatatattaaattcaaacatgactataatattttagaatttaacgatg
gtacattcgaatatggtgcacgtccacaatttaataaaccagcagcgaaa
actgatgcaactattaaaaaagaacaaaaattgattcaagctcaaaatct
tgtgagagaatttgaaaaaacacatactgtcagtgcacacagaaaagcac
aaaaggcagtcaacttagtttcgtttgaatacaaagtgaagaaaatggtc
ttacaagagcgaattgataatgtattaaaacaaggattagttaaataa

A number of homologous Staphylococcus Efb amino acid and nucleotide sequences are available from Genbank. Similarly, a number of homologous Efb amino acid and nucleotide sequences from other Staphylococcus species are also available from Genbank.

Any one or more of these Efb sequences, whether now known or hereafter identified, can be used in accordance with the present invention. In certain embodiments, the homologous amino acid sequences comprise at least about 80 percent identity, at least about 85 percent identity, at least about 90 percent identity, or at least about 95 percent identity to either the full length Efb amino acid sequence provided above, the N-terminal half of the Efb amino acid sequence provided above, or the C-terminal half of the Efb amino acid sequence provided above.

The use of Efb polypeptide fragments is also contemplated, including fragments containing either linear or conformational epitopes. Such fragments may include at least 20, at least 30, at least 40, at least 50, at least 60, at least 70, at least 80, at least 90, or at least 100 contiguous amino acids. Exemplary polypeptide fragments include those containing all or part of the N-terminal half of Efb, particularly the N-terminal fibrinogen binding domain, all or part of the C-terminal half of Efb, particularly the C-terminal fibrinogen binding domain, as well as those containing the region between the N-terminal and C-terminal fibrinogen binding domains.

Any one or more of the above-identified polypeptides can be synthesized by solid phase or solution phase peptide synthesis, recombinant expression, or can be obtained from natural sources. Automatic peptide synthesizers are commercially available from numerous suppliers, such as Applied Biosystems, Foster City, Calif. Standard techniques of chemical peptide synthesis are well known in the art (see e.g., SYNTHETIC PEPTIDES: A USERS GUIDE 93-210, Gregory A. Grant ed. (1992), which is hereby incorporated by reference in its entirety). Protein or polypeptide production via recombinant expression can be carried out using bacteria, such as E. coli, yeast, insect or mammalian cells and suitable expression systems. Procedures for recombinant protein/polypeptide expression are well known in the art and are described by Sambrook et al, Molecular Cloning: A Laboratory Manual, C.S.H.P. Press, NY 2d ed., (1989), which is hereby incorporated by reference in its entirety.

Recombinantly expressed polypeptides can be purified using any one of several methods readily known in the art, including ion exchange chromatography, hydrophobic interaction chromatography, affinity chromatography, gel filtration, and reverse phase chromatography. The polypeptide is preferably produced in purified form (preferably at least about 80% or 85% pure, more preferably at least about 90% or 95% pure) by conventional techniques. Depending on whether the recombinant host cell is made to secrete the polypeptide into growth medium (see U.S. Pat. No. 6,596,509 to Bauer et al., which is hereby incorporated by reference in its entirety), the polypeptide can be isolated and purified by centrifugation (to separate cellular components from supernatant containing the secreted polypeptide) followed by sequential ammonium sulfate precipitation of the supernatant. The fraction containing the polypeptide is subjected to gel filtration in an appropriately sized dextran or polyacrylamide column to separate the polypeptide from other proteins. If necessary, the polypeptide fraction may be further purified by HPLC and/or dialysis.

Affinity purification can also be utilized. For example, the recombinant DNA that encodes one of the above-identified polypeptides can be fused in-frame with a DNA sequence encoding a protein tag sequence that is useful for subsequent purification of the recombinantly expressed fusion polypeptide. Examples of protein tags are identified above. Once the recombinant fusion protein is recovered, a solution comprising the recombinant protein can be passed over a column designed specifically to retain protein comprising the tag. Upon elution, a substantially pure recombinant protein solution is obtained.

Once having obtained a substantially pure polypeptide, that polypeptide is then used to fabricate the diagnostic device.

In certain embodiments, the polypeptides are immobilized, permanently or reversibly, on a solid support such as a bead, chip, or slide. In certain embodiments, the immobilized polypeptides are arrayed and/or otherwise labeled for deconvolution of the binding data to yield identity of the immobilized polypeptide (and therefore of the antibody to which it binds) and, optionally, to quantitate binding.

Common solid supports include glass slides, silicon, microwells, nitrocellulose or PVDF membranes, and magnetic and other microbeads. While microdrops of protein delivered onto planar surfaces are widely used, related alternative architectures include CD centrifugation devices based on developments in microfluidics and specialized chip designs, such as engineered microchannels in a plate (The Living Chip™, Biotrove) and tiny 3D posts on a silicon surface (Zyomyx). Particles in suspension can also be used as the basis of arrays, providing they are coded for identification. Exemplary systems include, without limitation, color coding for microbeads (Luminex®, Bio-Rad) and semiconductor nanocrystals (QDots™, Quantum Dots), and barcoding for beads (UltraPlex™, Smartbeads) and multimetal microrods (Nanobarcodes™ particles, Surromed). Beads can also be assembled into planar arrays on semiconductor chips (LEAPS technology, BioArray Solutions).

The variables in immobilization of polypeptides include both the coupling reagent and the nature of the surface being coupled to. Ideally, the immobilization method used should be reproducible, applicable to polypeptides of different properties (size, hydrophilic, hydrophobic), amenable to high throughput and automation, and compatible with retention of polypeptide conformation and its epitopes.

The properties of a good protein array support surface are that it should be chemically stable before and after the coupling procedures, allow good spot morphology, display minimal nonspecific binding, not contribute a background in detection systems, and be compatible with different detection systems.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents (Telechem). In the Versalinx™ system (Prolinx), reversible covalent coupling is achieved by interaction between the protein derivatized with phenyldiboronic acid, and salicylhydroxamic acid immobilized on the support surface. This also has low background binding, low intrinsic fluorescence, and allows the immobilized proteins to retain function. Noncovalent binding of unmodified protein occurs within porous structures such as HydroGel™ (PerkinElmer), based on a 3-dimensional polyacrylamide gel; this substrate is reported to give a particularly low background on glass microarrays, with a high capacity and retention of protein function. Widely used biological capture methods are through biotin/streptavidin, having modified the protein appropriately to include, e.g., a C-terminal polypeptide fusion sequence such as GLNDIFEAQKIEWHE (SEQ ID NO: 24, AviTag™ sequence, GeneCopoeia, Inc.). Biotin may be conjugated to a fusion protein bearing such a fusion sequence by utilizing an appropriate recombinant host system for expression (e.g., BirA expressing E. coli).

By virtue of the polypeptides being bound to or present on a solid surface, they can be used to bind to antibodies present in a sample from an individual. The patient sample can be any type of sample as described above. The patient sample can be used in undiluted form or diluted form. Dilution from about 2:1 up to about 2500:1, particularly from about 20:1 to 1500:1 or 50:1 to 1000:1, is suitable for purposes of optimizing the read out of the diagnostic device and disclosed methods.

In certain embodiments, the immobilized polypeptides are used in a “sandwich” type assay in which a second, labeled antibody or binding fragment is used to bind specifically to any antibodies that are bound specifically by the immobilized polypeptides. Secondary antibodies used for labeling include, without limitation, anti-IgG antibodies, particularly anti-human IgG antibodies. A number of anti-IgG antibodies are commercially available, including without limitation the following products: anti-human IgG-FITC, anti-human IgG-PE, anti-human IgG-APC, and anti-human IgG-VioBlue, all of which are available from Miltenyi Biotec and are described as suitable for all classes of IgG antibodies; various Alexa Fluor® anti-human IgG antibody and QDot® anti-human IgG antibody, anti-human IgG-FITC antibody, anti-human IgG-PE antibody, and anti-human IgG-HRP antibody, all of which are available from Life Technologies.

In one example, an ELISA assay is performed using a multi-well format, with each well containing one of the disclosed polypeptides. The polypeptides specifically capture their respective antibody from the sample being tested, and then a labeled secondary antibody covalently coupled to an enzyme is used to quantify the presence of the bound secondary antibody (and, thus, the primary antibody bound from the sample) by determining with a spectrophotometer the fluorescence caused by a fluorescent label on the secondary antibody or chemiluminescence caused by an enzymatic label on the secondary antibody. Methods for performing ELISA are well known in the art and described in, for example, Perlmann, H. and Perlmann, P., Enzyme-Linked Immunosorbent Assay. In: Cell Biology: A Laboratory Handbook. San Diego, Calif., Academic Press, Inc., 322-328 (1994); Crowther, J. R., Methods in Molecular Biology, Vol. 42-ELISA: Theory and Practice, Humana Press, Totowa, N.J. (1995); and Harlow, E. and Lane, D., Antibodies: A Laboratory Manual. Cold Spring Harbor Laboratory Press, Cold Spring Harbor, N.Y., pp. 553-612 (1988), the contents of each of which are incorporated by reference in their entirety. Sandwich ELISAs for the quantitation of antibodies of interest are especially valuable when the concentration of the antibody of interest in the sample is low and/or the antibody of interest is present in a sample that contains high concentrations of other antibodies.

A fully-automated, microarray-based approach for high-throughput ELISAs is described by Mendoza et al. (BioTechniques 27:778-780, 782-786, 788 (1999), which is hereby incorporated by reference in its entirety). This system consists of an optically flat glass plate with 96 wells separated by a Teflon mask. More than a hundred capture molecules are immobilized in each well. Sample incubation, washing and fluorescence-based detection are performed using an automated liquid pipetter. The microarrays are quantitatively imaged with a scanning charge-coupled device (CCD) detector. Thus, the feasibility of multiplex detection of arrayed antigens in a high-throughput fashion using marker antigens is demonstrated. In addition, Silzel et al. (Clin Chem 44:2036-2043 (1998), which is hereby incorporated by reference in its entirety) demonstrates that multiple IgG subclasses can be detected simultaneously using microarray technology.

Most of the microarray assay formats described in the art rely on chemiluminescence- or fluorescence-based detection methods. A further improvement with regard to sensitivity involves the application of fluorescent labels and waveguide technology. A fluorescence-based array immunosensor was developed by Rowe et al. (Anal Chem 71:433-439 (1999); and Biosens Bioelectron 15:579-589 (2000), each of which is hereby incorporated by reference in its entirety) and applied for the simultaneous detection of clinical analytes using the sandwich immunoassay format and visualization with appropriate fluorescently labelled detection molecules. This array immunosensor was shown to be appropriate for the detection and measurement of targets at physiologically relevant concentrations in a variety of clinical samples.

A further increase in the sensitivity using waveguide technology was achieved with the development of the planar waveguide technology (Duveneck et al., Sens Actuators B B38:88-95 (1997), which is hereby incorporated by reference in its entirety). Thin-film waveguides are generated from a high-refractive material such as Ta₂O₅ that is deposited on a transparent substrate. Laser light of desired wavelength is coupled to the planar waveguide by means of diffractive grating. The light propagates in the planar waveguide and an area of more than a square centimeter can be homogeneously illuminated. At the surface, the propagating light generates a so-called evanescent field. This extends into the solution and activates only fluorophores that are bound to the surface. Fluorophores in the surrounding solution are not excited. Close to the surface, the excitation field intensities can be a hundred times higher than those achieved with standard confocal excitation. A CCD camera is used to identify signals simultaneously across the entire area of the planar waveguide. Thus, the immobilization of the capture molecules in a microarray format on the planar waveguide allows the performance of highly sensitive miniaturized and parallel immunoassays. This system was successfully employed to detect interleukin-6 at concentrations as low as 40 fM and as the additional advantage that the assay can be performed without washing steps that are usually required to remove unbound detection molecules (Weinberger et al., Pharmacogenomics 1:395-416 (2000), which is hereby incorporated by reference in its entirety).

Other immunoassays commonly used to quantitate the levels of proteins in cell samples are well-known in the art and can be adapted for use in the instant invention. The invention is not limited to a particular assay procedure, and therefore is intended to include both homogeneous and heterogeneous procedures. Exemplary other immunoassays which can be conducted according to the invention include fluorescence polarization immunoassay (FPIA), fluorescence immunoassay (FIA), and enzyme immunoassay (EIA). General techniques to be used in performing the various immunoassays noted above are known to those of ordinary skill in the art. In one embodiment, the determination of protein level in a biological sample may be performed by a microarray analysis (protein chip).

As an alternative to planar microarrays, bead-based assays combined with fluorescence-activated cell sorting (FACS) have been developed to perform multiplexed immunoassays. Fluorescence-activated cell sorting has been routinely used in diagnostics for more than 20 years.

Bead-based assay systems employ microspheres as solid support for the capture polypeptides instead of a planar substrate, which is conventionally used for microarray assays. In each individual immunoassay, the capture polypeptide is coupled to a distinct type of microsphere. The reaction takes place on the surface of the microspheres. The individual microspheres are color-coded by a uniform and distinct mixture of fluorescent dyes or intrinsically fluorescent materials that form the microspheres. After coupling of the appropriate capture polypeptide, i.e., a different capture polypeptide for each different types of bead, the different color coded bead sets can be pooled and the immunoassay is performed in a single reaction vessel. Recovery of an antibody from the sample being tested (i.e., specific binding of the antibody by a capture polypeptide) allows the different bead types to be detected with a fluorescence-based reporter system. The signal intensities are measured in a flow cytometer, which is able to quantify the amount of captured targets on each individual bead. Each bead type and thus each immobilized target is identified using the color code measured by a second fluorescence signal. This allows the multiplexed quantification of multiple targets from a single sample. Sensitivity, reliability and accuracy are similar to those observed with standard microtiter ELISA procedures. Color-coded microspheres can be used to perform up to a hundred different assay types simultaneously (LabMAP system, Laboratory Multiple Analyte Profiling, Luminex, Austin, Tex., USA). For example, microsphere-based systems have been used to simultaneously quantify cytokines or autoantibodies from biological samples (Carson and Vignali, J Immunol Methods 227:41-52 (1999); Chen et al., Clin Chem 45:1693-1694 (1999); Fulton et al., Clin Chem 43:1749-1756 (1997), each of which is hereby incorporated by reference in its entirety).

Bead-based systems have several advantages. As the capture polypeptides are coupled to distinct microspheres, each individual coupling event can be perfectly analyzed. Thus, only quality-controlled beads can be pooled for multiplexed immunoassays. Furthermore, if an additional parameter has to be included into the assay, one must only add a new type of loaded bead. No washing steps are required when performing the assay. The sample is incubated with the different bead types together with fluorescently labeled detection antibodies. After formation of the sandwich immuno-complex, only the fluorophores that are definitely bound to the surface of the microspheres are counted by the flow cytometer.

By way of example, one type of diagnostic device includes a plurality of LumAvidin™ beads (Luminex, Austin, Tex.), each of which has a distinct, biotinylated polypeptide tethered to its surface by the avidin bound to the bead surface. For example, Gmd-labeled beads, Amd-labeled beads, IsdA-labeled beads, IsdB-labeled beads, IsdH-labeled beads, ClfA-labeled beads, ClfB-labeled beads, FnbpA-labeled beads, SCIN-labeled beads, CHIPS-labeled beads, Hla-labeled beads, and Efb-labeled beads can be used to detect and quantify antibodies that bind specifically to these polypeptides or fragments thereof, as identified. Each of the plurality of beads has a distinct fluorescence pattern. As a consequence, the plurality of beads can be pooled into a single solution for exposure to a patient sample. Following exposure of the pooled bead solution, bound antibody can be labeled with anti-human IgG bearing a fluorescent label and then the labeled fluorescent beads can measured by flow cytometry. Fluorescent emissions due to the labeled anti-human IgG indicate a positive result, whereas the fluorescent emission of the bead identifies the specific polypeptide (e.g., Gmd, Amd, IsdA, IsdB, IsdH, ClfA, ClfB, FnbpA, SCIN, CHIPS, Hla, and Efb) used to prepare the functional bead and, hence, the specificity of the antibody that was bound to the bead. There is a quantitative relationship between level of fluorescent emissions and amount of target antibody bound by the polypeptide. For quantification, the antibody level of each antigen can be normalized to a positive control serum, and then each value derived relative to the median of the controls.

In several other embodiments, detection of the presence of an antibody in the sample upon its capture by the polypeptides arrayed onto a suitable surface and without labeling. For example, determining the ability of an antibody to bind to a capture polypeptide can be accomplished using a technology such as real-time Biomolecular Interaction Analysis (BIA). Sjolander, S. and Urbaniczky, C., Anal. Chem. 63:2338-2345 (1991); and Szabo et al., Curr. Opin. Struct. Biol. 5:699-705 (1995), each of which is hereby incorporated by reference in its entirety. As used herein, “BIA” is a technology for studying biospecific interactions in real time, without labeling any of the interactants (e.g., BIAcore).

In another embodiment, a biosensor with a special diffractive grating surface may be used to detect/quantitate binding between non-labeled antibodies in a biological sample and immobilized capture polypeptides at the surface of the biosensor. Details of the technology is described in more detail in B. Cunningham, P. Li, B. Lin, J. Pepper, Sensors and Actuators B, 81: 316-328 (2002); PCT Application Publ. No. WO 02/061429 A2; US Application Publ. No. 2003/0032039, each of which is hereby incorporated by reference in its entirety. Briefly, a guided mode resonant phenomenon is used to produce an optical structure that, when illuminated with collimated white light, is designed to reflect only a single wavelength (color). When molecules are attached to the surface of the biosensor, the reflected wavelength (color) is shifted due to the change of the optical path of light that is coupled into the grating. By linking capture polypeptides to the grating surface, captured antibodies can be detected/quantitated without the use of any kind of fluorescent probe or particle label. The spectral shifts may be analyzed to determine the expression data provided, and to indicate the presence or absence of a particular indication.

Label-free detection systems can be performed using any of a variety of sensors designed for use with Arrayed Imaging Reflectometry detection systems, Surface Plasmon Resonance detection systems, Brewster-Angle Straddle Interferometry detection systems, and ellipsometry detection systems, as well as any other label-free or fluorescence labeled array technique.

One example of an AIR detection system is described in U.S. Pat. No. 7,292,349 to Miller et al., which is hereby incorporated by reference in its entirety. This system includes a light source, a polarizer, a functionalized sensor chip of the present invention, and a detector. The light source generates and transmits light at a set wavelength towards a surface of the receptor. One or more lenses and filters can be employed to optimize the system. AIR exploits interference between reflections from the medium/coating and coating/substrate interfaces on the receptor, exhibiting changes in reflectivity upon binding of biomolecules to the coating. In practice, using a silicon wafer having an oxide coating, judicious choice of incident angle and wavelength can be used with s-polarized light to obtain near complete destructive interference (i.e., reflectivity that is preferably less than about 10⁻⁵ or even 10⁻⁶ under some circumstances) in the absence of a target, in this case the antibodies present in a sample. The condition of near complete (or near perfect) destructive interference is removed upon target binding. Thus, highly sensitive detection of even small quantities of antibodies is possible.

While AIR using s-polarized light has proven to be a highly sensitive, simple analytical method for the quantitative detection of a variety of biomolecular analytes, the system described in the above-referenced U.S. Pat. No. 7,292,349 to Miller et al. is much more easily carried out in a dry state, that is, with an air/oxide interface rather than with an aqueous/oxide interface. An improved system for performing AIR in an aqueous environment is described in U.S. Pat. No. 8,502,982 to Mace et al., which is hereby incorporated by reference in its entirety. Basically, the flow cell as described therein allows for coupling of the s-polarized light into the aqueous environment for detection of target binding. Use of this same flow cell, containing a sensor chip functionalized with the plurality of polypeptides specific for the antibodies described herein, is contemplated herein.

In both the wet and dry AIR systems, the sensor chip has the same fundamental construction, with a substrate, one or more coating layers on the substrate, and then the probe molecules—in this case the polypeptides—bound to discrete locations on the coating surface. As described in the above-referenced U.S. Pat. No. 7,292,349 to Miller et al. and U.S. Pat. No. 8,502,982 to Mace et al., a number of different materials can be selected for the substrate and coating(s). Any suitable combination of substrates and coatings is contemplated for the sensor chip to be used in an AIR detection system. Detection of serum antibodies using AIR has been demonstrated in U.S. Pat. No. 8,450,056 to Miller et al. and U.S. Pat. No. 8,486,619 to Miller et al., each of which is hereby incorporated by reference in its entirety.

The BASI detection system is described in U.S. Pat. No. 7,551,294 to Rothberg et al., which is hereby incorporated by reference in its entirety. The BASI system, like the AIR system, exploits interference between reflections from the medium/coating and coating/substrate interfaces, and exhibits changes in reflectivity upon binding of biomolecules to the coating. The basic design of the system is similar to that for AIR, but the structure of the sensor chip differs. The BASI system is functional with any substrate/coating combinations where the coating is very thin (e.g., a native oxide film on silicon) and when the incidence angle on one of two interfaces (substrate/coating or coating/medium) is greater than its Brewster angle and the incidence angle on the other of the two interfaces is less than its Brewster angle. Unlike AIR systems being commercially developed for use with incident s-polarized light, the BASI system relies on the detection with p-polarized light. As a result of using Brewster angle straddle and p-polarized light, where the coating thickness is <<λ, a phase flip of the reflected polarization allows nearly complete destructive interference (where reflectivity is preferably less than about 10⁻⁴ or even 10⁻⁵ in the absence of target binding). As with the AIR detection system, sensitive detection of even small quantities of antibodies is possible.

Ellipsometric detection systems measure the polarization component of reflected light as a measure of changes in coating thickness on the surface of the sensor chip. Ellipsometry sensitively measures the change of the state of polarization when electromagnetic radiation is reflected or transmitted by a sample. A classical embodiment of such an ellipsometric detection system includes a light source that emits a collimated light beam passing a variable polarization controller given by the combination of a linear polarizer and a compensator in the form of a quarter-wave plate. The polarized light beam is incident on the sensor surface under a known oblique angle, reflected from the sample surface and analyzed by a second linear polarizer coupled to a suitable photodetector. Imaging ellipsometry, as described for example in U.S. Pat. No. 5,076,696 to Cohn et al., which is hereby incorporated by reference in its entirety, uses spatially resolving detector and imaging optics to allow for a massively parallel measurement of ellipsometric data, e.g., in the form of Delta and/or Psi maps. Such maps may in turn be converted into surface maps of layer thickness, optical index of refraction, chemical composition or the amount of adsorbed material for each spot on an array. Imaging ellipsometry with its intrinsic parallel detection scheme may be used advantageously as a detection technique for these so-called biochips, microarrays or microplates (Eing et al., Imaging Ellipsometry in Biotechnology, ISBN 3-9807279-6-3 (2002), which is hereby incorporated by reference in its entirety). Imaging ellipsometry has been demonstrated with light employed for the measurement impinging on the surface to be measured coming from the ambient medium. Other measurement setups are based on total internal reflection as described for example in U.S. Pat. No. 6,594,011 to Kempen, which is hereby incorporated by reference in its entirety. Here, the light from a light source is directed through an internal reflection element to reflect off the specimen to be detected.

Enhancement of the detection signal can be achieved using SPR ellipsometry. The substrate employed during SPR ellipsometry uses a thin metal layer to allow the excitation and propagation of surface plasmons. While one side of the metal layer is in contact with a transparent support structure, usually attached to a prism allowing light to couple-in under an oblique angle, the other side of the layer is exposed to the ambient medium. Changes in the optical index of refraction in the ambient by the formation of an adsorbent layer (e.g., antibodies from the sample binding to surface-bound polypeptides) are monitored as a shift in the angle of incidence that generates surface plasmon resonance, causing a change of reflected light intensity. For SPR based sensors it is known that an intermediate dielectric layer between the metal film and the probed surface may act as a means to further increase the sensitivity. One exemplary SPR substrate is described in U.S. Pat. No. 7,332,329 to Wark et al., which is hereby incorporated by reference in its entirety. This SPR substrate is particularly suited for biomolecular arrays of polypeptides, where the substrate includes a plurality of a metallic islands surrounded by a hydrophobic layer or a dielectric material, and the polypeptides are bound to the metallic islands.

The diagnostic device may also be constructed in the format of a lateral flow diagnostic device. In such a device, the antibodies in the sample bind specifically to the polypeptide of interest which is present on the detection strip. Once the antibodies from the sample adhere on this antigen, a marked or labeled secondary antibody binds on said antibody from the sample. Marking of the antibody-antigen complex, in this case with gold nanoparticles, quantum dots, or the like, makes the strip appear colored as soon as enough marked antibodies have bound. Examples of such lateral flow test devices are well known in the art, and include, without limitation, U.S. Application Publ. No. 20130022965 to Von Olleschikelbheim et al., U.S. Application Publ. No. 20110201131 to Badwan et al., U.S. Application Publ. No. 20110143365 to Buchanon, U.S. Application Publ. No. 20110091906 to Ford et al., U.S. Application Publ. No. 20080254441 to Mohammed, and U.S. Application Publ. No. 20070243630 to Beohringer et al., each of which is hereby incorporated by reference in its entirety. In one embodiment, a lateral flow diagnostic device includes an IsdB polypeptide present on the test strip for detection of IsdB antibodies present in the sample.

Regardless of the format, the support surface containing the capture polypeptides of interest are exposed individually or simultaneously to the biological sample using conditions suitable to allow for the specific binding of any antibodies in the sample to the surface-bound polypeptides. Label-free detection of the specific binding event can be carried out using the appropriate assay protocol as described above. Detection and quantification of the antibody is based upon the changed read-out of the label-free detection parameters, including without limitation a change in reflectivity of incident light, a shift in the wavelength of incident light, a change in the intensity of output light, etc., as described above. Alternatively, a secondary antibody can be exposed to the substrate and allowed to react with any antibody captured by the surface-bound polypeptides. Detection and quantification of the antibody is based upon the degree of label measured from the secondary antibody.

Using the various embodiments to measure the presence of antibodies in the biological sample being analyzed, the presence of an active Staphylococcus infection can be determined by the presence of antibodies (specific for one or more of the Staphylococcus polypeptides) in the sample being screened.

In one embodiment, the diagnostic device contains IsdB polypeptide and the specific binding of the IsdB polypeptide to antibodies present in a sample obtained from an individual indicates the presence of an active Staphylococcus infection in the individual from whom the sample was obtained.

In another embodiment, the diagnostic device contains a plurality of the above-identified polypeptides, including any two or more, any three or more, any four or more, any five or more, any six or more, any seven or more, any eight or more, any nine or more, any ten or more, any eleven or more, or all twelve of the proteins or polypeptides of Gmd, Amd, IsdA, IsdB, IsdH, ClfA, ClfB, FnbpA, SCIN, CHIPS, Hla, and Efb. In one exemplary embodiment, the diagnostic device contains at least the proteins or polypeptides of IsdB, IsdH, HLA, and SCIN. In another exemplary embodiment, the diagnostic device contains at least the proteins or polypeptides of IsdB, Gmd, Amd, IsdA, IsdH, ClfA, and ClfB. In yet another exemplary embodiment, the diagnostic device contains the proteins or polypeptides of IsdA, IsdB, IsdH, ClfB, SCIN, CHIPS, Hla, and Efb. In an alternative exemplary embodiment, the diagnostic device contains at least the proteins or polypeptides of Gmd, Amd, IsdA, IsdB, IsdH, ClfA, ClfB, SCIN, CHIPS, Hla, and Efb.

If a threshold number of positive polypeptide-antibody binding events are detected, then this indicates the presence of an active Staphylococcus infection in the individual from whom the sample was obtained.

For example, in certain embodiments the threshold number can be binding events for at least three distinct types of antibodies selected from the group of anti-Gmd antibodies, anti-Amd antibodies, anti-IsdA antibodies, anti-IsdB antibodies, anti-IsdH antibodies, anti-ClfA antibodies, anti-ClfB antibodies, anti-FnbpA antibodies, anti-SCIN antibodies, anti-CHIPS antibodies, anti-Hla antibodies, and anti-Efb antibodies. Preferably, one of the at least three distinct types of antibodies is anti-IsdB antibody. In certain embodiments, others of the at least three distinct types of antibodies are selected from anti-Gmd antibodies, anti-Amd antibodies, anti-IsdA antibodies, anti-IsdB antibodies, anti-IsdH antibodies, anti-ClfA antibodies, and anti-ClfB antibodies. In certain other embodiments, others of the at least three distinct types of antibodies are selected from anti-IsdH antibodies, anti-Hla antibodies, and anti-SCIN antibodies.

In another example, the threshold number can be binding events for at least five distinct types of antibodies selected from the group of anti-Gmd antibodies, anti-Amd antibodies, anti-IsdA antibodies, anti-IsdB antibodies, anti-IsdH antibodies, anti-ClfA antibodies, anti-ClfB antibodies, anti-FnbpA antibodies, anti-SCIN antibodies, anti-CHIPS antibodies, anti-Hla antibodies, and anti-Efb antibodies. Preferably, one of the at least five distinct types of antibodies is anti-IsdB antibody. In certain embodiments, others of the at least five distinct types of antibodies are selected from anti-Gmd antibodies, anti-Amd antibodies, anti-IsdA antibodies, anti-IsdB antibodies, anti-IsdH antibodies, anti-ClfA antibodies, and anti-ClfB antibodies. In other embodiments, others of the at least five distinct types of antibodies include anti-IsdH antibodies, anti-Hla antibodies, and anti-SCIN antibodies.

In yet another example, the threshold number can be binding events for at least seven distinct types of antibodies selected from the group of anti-Gmd antibodies, anti-Amd antibodies, anti-IsdA antibodies, anti-IsdB antibodies, anti-IsdH antibodies, anti-ClfA antibodies, anti-ClfB antibodies, anti-FnbpA antibodies, anti-SCIN antibodies, anti-CHIPS antibodies, anti-Hla antibodies, and anti-Efb antibodies. Preferably, one of the at least seven distinct types of antibodies is anti-IsdB antibody. In certain embodiments, the at least seven distinct types of antibodies include anti-Gmd antibodies, anti-Amd antibodies, anti-IsdA antibodies, anti-IsdB antibodies, anti-IsdH antibodies, anti-ClfA antibodies, and anti-ClfB antibodies. In other embodiments, others of the at least seven distinct types of antibodies include anti-IsdH antibodies, anti-Hla antibodies, and anti-SCIN antibodies.

A further aspect of the disclosure relates to the use of a panel of antigen to identify a joint replacement patient having a higher likelihood of needing revision joint replacement surgery. The diagnostic procedure described above is performed on a sample obtained from a patient that received a total joint replacement and, therefore, is at risk of infection. The sample is taken to identify whether (or not) the patient has an active Staphylococcus infection; if an active Staphylococcus infection is identified, based on the level of anti-Amd, anti-Gmd, or anti-ClfB antibodies (or any combination thereof) as measured during the initial diagnostic procedure, it is next determined whether the level of one or more of those antibody levels, while perhaps elevated, is lower than a therapeutically-effective threshold titer. An anti-Amd titer, an anti-Gmd titer, an anti-ClfB titer, or any combination thereof, which is lower than a therapeutically-effective threshold titer indicates that the patient is likely to need revision joint replacement surgery. Threshold titers can be determined from the ROC curve to maximize clinical impact (i.e., maximize sensitivity or specificity in accordance with clinical need). Adjustment of the threshold titer in this regard is illustrated in the accompanying Examples (albeit for detection of active infection).

The outcome of the inventive diagnostic procedure can, of course, provide an opportunity to intervene with therapeutic treatment of the active Staphylococcus infection at an earlier point in time than would otherwise have been possible. For example, it is possible to administer to the patient antibiotics, a passive vaccine comprising an anti-Amd monoclonal antibody, an anti-Gmd monoclonal antibody, an anti-ClfB monoclonal antibody, or any combination of two or more of those antibodies; or both passive vaccine(s) and antibiotic agents. Exemplary passive vaccines are described in PCT Application Publ. No. WO/2013/066876 and WO/2011/140114, both to Schwarz et al., PCT Application No. PCT/US14/70337 to University of Rochester et al., filed Dec. 15, 2014, and U.S. Pat. No. 6,680,195 to Patti et al., each of which is hereby incorporated by reference in its entirety.

In addition to the diagnostic devices and their use, the present invention also includes the use of these diagnostic devices in kits with one or more reagents for the detection of active Staphylococcus infections. The kits may include any of the diagnostic devices described above as well as any one or more of a secondary antibody having label, one or more buffer solutions (e.g., reaction buffer, wash buffer, etc.), an enzymatic substrate solution, and a sample collection device. Also included are instructions for the use of these reagents for the detection in patient samples of antibodies that recognize (i.e., bind specifically) to polypeptides corresponding to Staphylococcus proteins of the types described herein.

The Example set forth below is for illustrative purposes only and is not intended to limit, in any way, the scope of the claimed invention.

### EXAMPLE 1

### Detection of Active S. aureus Infection

All studies with human subjects and vertebrate animals were performed on IRB approved protocols. The 12 S. aureus antigens selected, based on their established immunogenicity and pathogenic roles, are listed in Table 1 below.

TABLE 1
List of Antigen and Their Function

| Function | Name of Antigen | Abbreviation |
|---|---|---|
| Enzyme involved | Glucosaminidase | Gmd |
| in cell division | Amidase | Amd |
| Iron scavenging | Iron-regulated surface determinant | IsdA |
| protein | protein A | |
| | Iron-regulated surface determinant | IsdB |
| | protein B | |
| | Iron-regulated surface determinant | IsdH |
| | protein H | |
| Cell wall adhesin | Clumping Factor A | ClfA |
| | Clumping Factor B | ClfB |
| | Fibronectin Binding Protein A | FnbpA |
| Secreted | Staphylococcus Complement Inhibitor | SCIN |
| virulence factors | Chemotaxis Inhibitory Protein of | CHIPS |
| | Staph. aureus | |
| | α-Hemolysin | Hla |
| | Extracellular Fibrinogen-binding Protein | Efb |

The entire DNA encoding region for each protein was synthesized de novo with a hexa-His tag on the N-terminus, and a 15 amino acid biotinylation sequence (AviTag™ sequence of SEQ ID NO: 24) at the C-terminus (Predonzani et al., BMC Biotechnol. 8:41 (2008); Beckett et al., Protein Science 8:921-929 (1999), each of which is hereby incorporated by reference in its entirety). The DNA sequence encoding the AviTag™ sequence of SEQ ID NO: 24 is as follows:

(SEQ ID NO: 25)

ggcctgaatg acatctttga agcacagaaa atcgaatggc acgaa

Recombinant proteins for the 12 antigens were produced in E. coli that express biotin ligase (BirA), which biotinylated the C-terminus of the antigen, and were purified by metal chelation chromatography. These purified recombinant antigens were validated via SDS-PAGE, western blotting and specific functional assays. The antigens were also evaluated for their ability to detect antibodies in sera obtained from Balb/c mice challenged with S. aureus, using sera from naïve mice as controls. Human sera were obtained from 32 patients (21 post TJR infections, 6 hardware infections after fracture surgeries, and 5 deep musculoskeletal infections without implant) with confirmed S. aureus deep musculoskeletal infections (Patients), and 40 non-infected patients prior to elective primary TJA surgery (Controls).

Antibody levels against the antigens were determined via Luminex assay. Briefly, unique LumAvidin™ beads (dual fluorescent bead covalently linked to streptavidin, Luminex, Austin, Tex.) for each antigen were separately coupled to assigned recombinant protein and washed. Then the antigen-laden beads were pooled together and incubated with serial dilutions of the individual human sera (starting at 1:100) in a 96 well plate for 2 hours, incubated with phycoerythrin conjugated (PE) goat anti-human total IgG for 1 hour, and then the fluorescent intensity of the beads and PE were measured with a flow cytometer (Bio-Plex 200, Bio-Rad). The accuracy of multiplex antigen measurement was validated by comparison with single antigen measurement using the same serum. For quantification, the antibody level of each antigen was normalized to a positive control serum, and then each value was derived relative to the median of the Controls sera.

Conventional ELISA results demonstrated that sera from the experimentally infected mice contained higher antibody titers vs. naïve mice for all the antigens. These results were replicated using the Luminex assay. There was no difference in the demographic information between Controls and Patients.

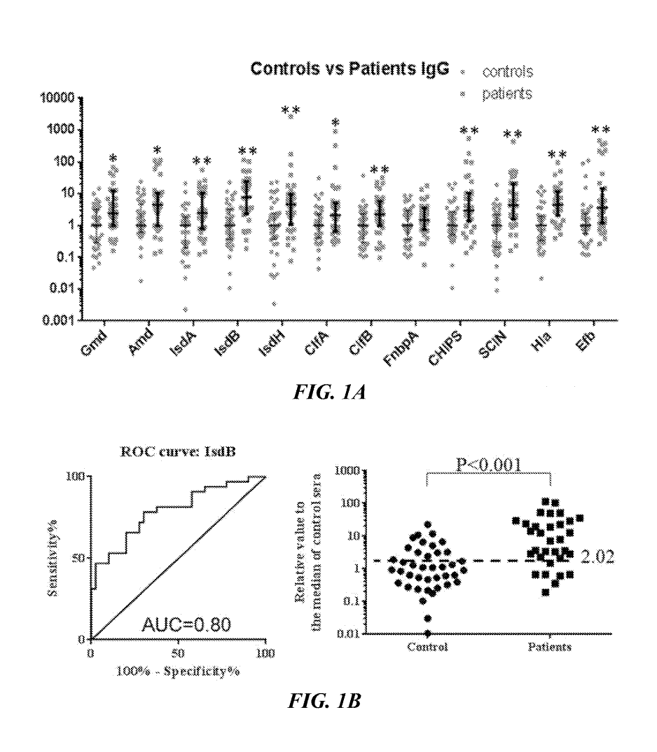

The results from the Luminex assay of the human sera are presented in FIG. 1, and demonstrate that antibody levels against Gmd, Amd, IsdA, IsdB, IsdH, ClfA, ClfB, SCIN, CHIPS, Hla, Efb were significantly higher in the Patients versus Controls sera. Only FnbpA did not show a statistically significant difference. Interestingly, the area under the curve (AUC) analysis of the Receiver Operating Characteristic (ROC) curve showed that IsdB had the greatest diagnostic value (0.80); and its sensitivity, specificity and positive predictive value (PPV) were 0.80, 0.70 and 0.68 respectively, using a cutoff value of 2.02 (FIG. 1B). The multivariate logistic regression of the 8 antibody levels (Gmd, Amd, IsdA, IsdB, IsdH, FnbpA, ClfA, and ClfB) combined demonstrated an AUC of 0.83 with diagnostic power of 71% sensitivity, 88% specificity and 82% PPV, using a cutoff value of 1.49 (FIG. 2). Using a more stringent cutoff value of 2.04, the multivariate analysis identified 60% of the infected patients with no false positives.

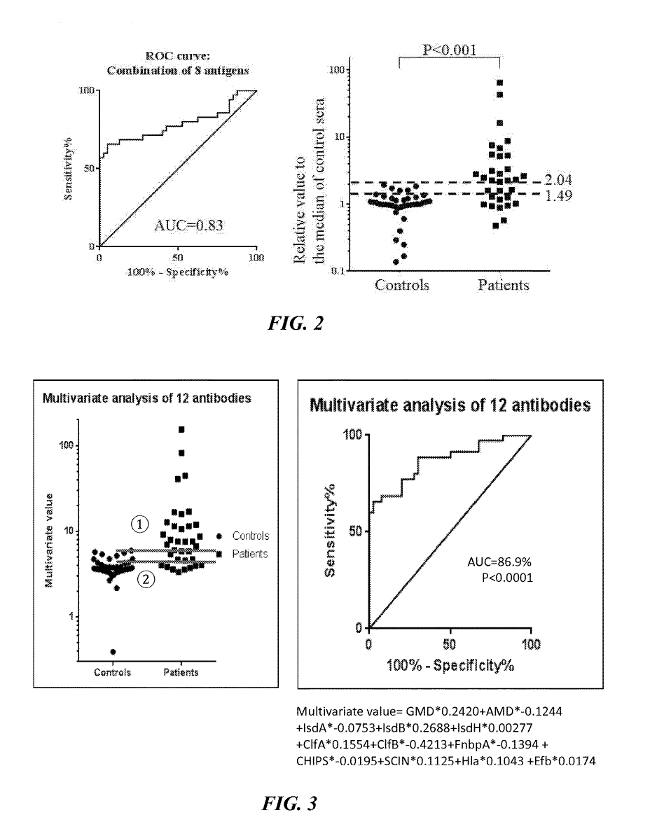

The multivariate logistic regression of all 12 antibody levels combined demonstrated an AUC of 0.87 with diagnostic power of 77.1% sensitivity, 80.0% specificity and 77.1% PPV, using a cutoff value of 4.42 (FIG. 3). Using a more stringent cutoff value of 5.99, the multivariate analysis identified 60% of the infected patients with no false positives.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. Additionally, the recited order of processing elements or sequences is not intended to limit the claimed processes to any order except as may be specified in the claims. These alterations, improvements, and modifications are intended to be suggested hereby, and are within the spirit and scope of the disclosure. Accordingly, the invention is limited only by the following claims and equivalents thereto.

1. A diagnostic device comprising:
a substrate comprising a plurality of discrete sites and one of a plurality of polypeptides present at each of the plurality of discrete sites, each of the polypeptides comprising an epitope that binds specifically to an antibody present in a sample from an individual having an active Staphylococcus infection,
wherein, upon exposure to the sample of an individual, the specific binding of a threshold number of the plurality of polypeptides to antibodies in the sample indicates the presence of an active Staphylococcus infection.

2. The diagnostic device according to claim 1, wherein the Staphylococcus infection is caused by a Staphylococcus strain selected from the group consisting of S. aureus, S. epidermidis, S. lugdunensis, S. saprophyticus, S. haemolyticus, S. caprae, and S. simiae.

3. (canceled)

4. The diagnostic device according to claim 1, wherein the plurality of polypeptides comprise three or more of polypeptides selected from the group of a glucosaminidase (Gmd) protein or polypeptide, an amidase (Amd) protein or polypeptide, an iron-regulated surface determinant protein A (IsdA) protein or polypeptide, an iron-regulated surface determinant protein B (IsdB) protein or polypeptide, an iron-regulated surface determinant protein H (IsdH) protein or polypeptide, a Clumping Factor A (ClfA) protein or polypeptide, a Clumping Factor B (ClfB) protein or polypeptide, a Fibronectin Binding Protein A (FnbpA) protein or polypeptide, a Staphylococcus Complement Inhibitor (SCIN) protein or polypeptide, a Chemotaxis Inhibitory Protein of Staphylococcus aureus (CHIPS) protein or polypeptide, an α-Hemolysin (Hla) protein or polypeptide, and an Extracellular Fibrinogen-binding (Efb) protein or polypeptide.

5. The diagnostic device according to claim 4, wherein the plurality of polypeptides comprise:
(i) IsdB protein or polypeptide, IsdH protein or polypeptide, SCIN protein or polypeptide, and Hla protein or polypeptide; or
(ii) IsdA protein or polypeptide, IsdB protein or polypeptide, IsdH protein or polypeptide, ClfB protein or polypeptide, SCIN protein or polypeptide, CHIPS protein or polypeptide, Hla protein or polypeptide, and Efb protein or polypeptide; or
(iii) Gmd protein or polypeptide, Amd protein or polypeptide, IsdA protein or polypeptide, IsdB protein or polypeptide, IsdH protein or polypeptide, ClfA protein or polypeptide, ClfB protein or polypeptide, SCIN protein or polypeptide, CHIPS protein or polypeptide, Hla protein or polypeptide, and Efb protein or polypeptide; or
(iv) Gmd protein or polypeptide, Amd protein or polypeptide, IsdA protein or polypeptide, IsdB protein or polypeptide, IsdH protein or polypeptide, ClfA protein or polypeptide, ClfB protein or polypeptide, FnbpA protein or polypeptide, SCIN protein or polypeptide, CHIPS protein or polypeptide, Hla protein or polypeptide, and Efb protein or polypeptide.

6. The diagnostic device according to claim 1, wherein the plurality of polypeptides comprise five or more of polypeptides selected from the group of Gmd protein or polypeptide, Amd protein or polypeptide, IsdA protein or polypeptide, IsdB protein or polypeptide, IsdH protein or polypeptide, ClfA protein or polypeptide, ClfB protein or polypeptide, FnbpA protein or polypeptide, SCIN protein or polypeptide, CHIPS protein or polypeptide, Hla protein or polypeptide, and Efb protein or polypeptide.

7. (canceled)

8. The diagnostic device according to claim 1, wherein the plurality of polypeptides comprise seven or more of polypeptides selected from the group of Gmd protein or polypeptide, Amd protein or polypeptide, IsdA protein or polypeptide, IsdB protein or polypeptide, IsdH protein or polypeptide, ClfA protein or polypeptide, ClfB protein or polypeptide, FnbpA protein or polypeptide, SCIN protein or polypeptide, CHIPS protein or polypeptide, Hla protein or polypeptide, and Efb protein or polypeptide.

9. (canceled)

10. (canceled)

11. The diagnostic device according to claim 1, wherein the substrate comprises a multiwell plate and each of the discrete sites comprises a well of the multiwell plate.

12. The diagnostic device according to claim 1, wherein the plurality of polypeptides are covalently bound to the discrete sites of the substrate or are noncovalently bound to the discrete sites of the substrate, wherein, when noncovalently bound to the discrete sites of the substrate, the plurality of peptides each comprise a biotin label and the substrate comprises avidin or streptavidin covalently bound to the discrete sites or is bound to an avidin- or streptavidin-labeled bead.

13-15. (canceled)

16. The diagnostic device according to claim 1, wherein each of the plurality of polypeptide is bound to a discrete bead, wherein each of the beads labeled with a particular polypeptide comprises a fluorescent property that is distinct of other beads labeled with a different polypeptide.

17. (canceled)

18. The diagnostic device according to claim 1, wherein the substrate comprises an antireflective coating or comprises surface-plasmon enhancing layer, diffraction grating, or waveguide.

19. (canceled)

20. The diagnostic device according to claim 1, wherein the threshold number is at least three.

21-28. (canceled)

29. A method of detecting an active Staphylococcus infection in an individual comprising:
obtaining a sample from an individual;
exposing the obtained sample to a plurality of polypeptides bound to a surface, each of the polypeptides comprising an epitope that binds specifically to an antibody present in serum of an individual having an active Staphylococcus infection; and
determining whether, after said exposing, the specific binding of a threshold number of the plurality of polypeptides to antibodies in the sample occurred, thereby indicating the presence of an active Staphylococcus infection.

30. The method according to claim 29, wherein said determining comprises quantifying the antibodies present in the obtained sample that are specific for each of the plurality of polypeptides, and comparing the quantified antibodies in the obtained sample with the quantified antibodies present in a control standard or an uninfected individual.

31. The method according to claim 30, wherein said exposing and said determining is carried out for the control standard or the sample from an uninfected individual in parallel with the sample from the individual.

32-41. (canceled)

42. The method according to claim 29, wherein the threshold number is at least three of the following polypeptides: Gmd protein or polypeptide, Amd protein or polypeptide, IsdA protein or polypeptide, IsdB protein or polypeptide, IsdH protein or polypeptide, ClfA protein or polypeptide, ClfB protein or polypeptide, FnbpA protein or polypeptide, SCIN protein or polypeptide, CHIPS protein or polypeptide, Hla protein or polypeptide, and Efb protein or polypeptide.

43. The method according to claim 42, wherein the at least three polypeptides comprise:
(i) IsdB protein or polypeptide, IsdH protein or polypeptide, SCIN protein or polypeptide, and Hla protein or polypeptide; or
(ii) IsdA protein or polypeptide, IsdB protein or polypeptide, IsdH protein or polypeptide, ClfB protein or polypeptide, SCIN protein or polypeptide, CHIPS protein or polypeptide, Hla protein or polypeptide, and Efb protein or polypeptide; or
(iii) Gmd protein or polypeptide, Amd protein or polypeptide, IsdA protein or polypeptide, IsdB protein or polypeptide, IsdH protein or polypeptide, ClfA protein or polypeptide, ClfB protein or polypeptide, SCIN protein or polypeptide, CHIPS protein or polypeptide, Hla protein or polypeptide, and Efb protein or polypeptide; or
(iv) Gmd protein or polypeptide, Amd protein or polypeptide, IsdA protein or polypeptide, IsdB protein or polypeptide, IsdH protein or polypeptide, ClfA protein or polypeptide, ClfB protein or polypeptide, FnbpA protein or polypeptide, SCIN protein or polypeptide, CHIPS protein or polypeptide, Hla protein or polypeptide, and Efb protein or polypeptide.

44. The method according to claim 29, wherein the threshold number is at least five of the following polypeptides: Gmd protein or polypeptide, Amd protein or polypeptide, IsdA protein or polypeptide, IsdB protein or polypeptide, IsdH protein or polypeptide, ClfA protein or polypeptide, ClfB protein or polypeptide, FnbpA protein or polypeptide, SCIN protein or polypeptide, CHIPS protein or polypeptide, Hla protein or polypeptide, and Efb protein or polypeptide.

45. (canceled)

46. The method according to claim 29, wherein the threshold number is at least seven of the following polypeptides: Gmd protein or polypeptide, Amd protein or polypeptide, IsdA protein or polypeptide, IsdB protein or polypeptide, IsdH protein or polypeptide, ClfA protein or polypeptide, ClfB protein or polypeptide, FnbpA protein or polypeptide, SCIN protein or polypeptide, CHIPS protein or polypeptide, Hla protein or polypeptide, and Efb protein or polypeptide.

47-52. (canceled)

53. A diagnostic device comprising:
a substrate comprising an Iron-regulated surface determinant protein B (IsdB) polypeptide present on the substrate, wherein the IsdB polypeptide comprises an epitope that binds specifically to an antibody present in serum of an individual having an active Staphylococcus infection,
wherein, upon exposure to the serum of an individual, the specific binding of the IsdB polypeptide to antibodies in the serum indicates the presence of an active Staphylococcus infection.

54. The diagnostic device according to claim 53, wherein the Staphylococcus infection is caused by a Staphylococcus strain selected from the group consisting of S. aureus, S. epidermidis, S. lugdunensis, S. saprophyticus, S. haemolyticus, S. caprae, and S. simiae.

55. (canceled)

56. The diagnostic device according to claim 53, wherein the device consists of the substrate and the IsdB polypeptide.

57. The diagnostic device according to claim 53, wherein the substrate comprises a multiwell plate.

58. The diagnostic device according to claim 53, wherein the IsdB polypeptide is covalently bound to the substrate or noncovalently bound to the substrate, wherein, when noncovalently bound to the substrate, the IsdB polypeptide comprises a biotin label and the substrate comprises avidin or streptavidin covalently bound thereto or is bound to an avidin- or streptavidin-labeled bead.

59-61. (canceled)

62. The diagnostic device according to claim 53, wherein the substrate comprises an antireflective coating or a surface-plasmon enhancing layer, diffraction grating, or waveguide.

63-69. (canceled)

70. A method of detecting an active Staphylococcus infection in an individual comprising:
obtaining a sample from an individual;
exposing the obtained sample to an IsdB polypeptide bound to a surface, the IsdB polypeptide comprising an epitope that binds specifically to an antibody present in serum of an individual having an active Staphylococcus infection; and
determining whether, after said exposing, the specific binding of the IsdB polypeptide to antibodies in the sample occurred, thereby indicating the presence of an active Staphylococcus infection.

71. The method according to claim 70, wherein said determining comprises quantifying the antibodies present in the obtained sample that are specific for the IsdB polypeptide, and comparing the quantified antibodies in the obtained sample with the quantified antibodies present in a control standard or an uninfected individual.

72. The method according to claim 70, wherein said exposing and said determining is carried out for the control standard or the sample from an uninfected individual in parallel with the sample from the individual.

73-84. (canceled)

85. A method of identifying a joint replacement patient having a higher likelihood of needing revision joint replacement surgery, the method comprising:
performing the method according to claim 29 to identify a patient that received a total joint replacement and has an active Staphylococcus infection;
determining whether the level of anti-Amd, anti-Gmd, or anti-ClfB antibodies, as measured during said performing, is lower than a threshold titer;
wherein an anti-Amd titer, an anti-Gmd titer, an anti-ClfB titer, or any combination thereof, that is lower than a threshold titer level indicates that the patient is likely to need revision joint replacement surgery.

86. The method according to claim 85 further comprising:
administering to the patient a passive vaccine comprising an anti-Amd monoclonal antibody, an anti-Gmd monoclonal antibody, an anti-ClfB monoclonal antibody, or a combination thereof; or
administering to the patient an antibiotic agent suitable for treating a Staphylococcus infection.

87. (canceled)
